(19) United States
(12) Patent Application Publication
(10) Pub. No.: US 2024/0123054 A1
(43) Pub. Date: Apr. 18, 2024

(54) CORONAVIRUS ANTIGENS AND EPITOPES AND PROTEINS THAT BIND THERETO

(71) Applicants: Joseph COTROPIA, Heath, TX (US); Gaurav Chandra, Heath, TX (US)

(72) Inventors: Joseph COTROPIA, Heath, TX (US); Gaurav Chandra, Heath, TX (US)

(21) Appl. No.: 18/277,536

(22) PCT Filed: Feb. 18, 2022
(86) PCT No.: PCT/US2022/016926

§ 371 Date: Aug. 16, 2023

Related U.S. Application Data
(60) Provisional application No. 63/152,084, filed on Feb. 22, 2021.

(51) Int. Cl. A61K 39/215; C07K 16/10

(52) U.S. Cl. CPC A61K 39/215; C07K 16/1003; C07K 2317/34; C07K 2317/76

(57) ABSTRACT

Provided are highly conserved antigens and epitopes of SARS-CoV-2 that can be used in vaccines and to produce bindings proteins (e.g., antibodies) for treating, preventing, or reducing the risks of infections caused by β-coronaviruses such as SARS-CoV-2, and as targets for detecting SARS-CoV-2 infection

### CROSS REFERENCE TO RELATED APPLICATIONS

This application claims priority under 35 U.S.C. § 119(e) to U.S. Provisional Application 63/152,084, filed Feb. 22, 2021, the entire contents of which are incorporated herein by reference in their entirety.

### FIELD

The present disclosure relates to the field of viral vaccines, therapeutics, and diagnostics, and in particular, discloses antigens and epitopes of SARS-CoV-2, the causative agent of COVID-19, that can be used in vaccines and to produce binding proteins (e.g., antibodies) for treating, preventing, or reducing the risks of infections caused by β-coronaviruses such as SARS-CoV-2, which antigens and binding proteins also can be used in assays and kits for detecting infection by β-coronaviruses such as SARS-CoV-2.

### BACKGROUND

The following discussion is merely provided to aid the reader in understanding the disclosure and is not admitted to describe or constitute prior art thereto.

The epidemic of novel coronavirus disease 2019 (COVID-19) was caused by a new coronavirus named severe acute respiratory syndrome coronavirus 2 (SARS-CoV-2), which has spread worldwide and turned into a global pandemic. SARS CoV-2 is a β-coronavirus and the seventh known coronavirus to infect humans. Four of these coronaviruses (229E, NL63, OC43, and HKU1) only cause slight symptoms of the common cold, while the other three (SARS-CoV, MERS-CoV, and SARS-CoV-2) can cause severe symptoms and even death.

SARS-CoV-2 is a single-stranded RNA-enveloped virus. An RNA-based metagenomic next-generation sequencing approach has been applied to characterize its entire genome, which is 29,881 bp in length (GenBank no. MN908947), encoding 9860 amino acids. Gene fragments express structural and nonstructural proteins. The S, E, M, and N genes encode structural proteins, whereas nonstructural proteins, such as 3-chymotrypsin-like protease, papain-like protease, and RNA-dependent RNA polymerase, are encoded by the ORF region. A large number of glycosylated S proteins cover the surface of SARS-CoV-2 and bind to the host cell receptor angiotensin-converting enzyme 2 (ACE2), mediating viral cell entry.

Numerous SARS-CoV-2 variants have emerged. The variants known as B.1.1.7 (Alpha) (first identified in the United Kingdom), B.1.351 (Beta) (first identified in South Africa), P.1 (Gamma) (first identified in Brazil), B.1.167.2 (Delta) (first identified in India), and B.1.1.529 (Omicron) (first identified in South Africa) concern scientists because of emerging data suggesting their increased ability to spread, ability to evade detection by specific viral diagnostic tests, decreased susceptibility to therapeutic agents, and ability to evade natural or vaccine-induced immunity. Variants can carry several different mutations, but changes in the spike protein of the virus, used to enter cells and infect them, are especially concerning as changes to this protein may cause a vaccine developed against the spike protein to be less effective against a particular variant.

SARS-CoV-2 variants of concern

| | Country First | Date | Spike Protein |
|---|---|---|---|
| Variant Name | Identified | Discovered | Changes of Concern |
| B.1.1.7 | UK | September 2020 | Del69-70 |
| (Alpha) | | | |
| B.1.351 | South Africa | October 2020 | E484K |
| (Beta) | | | N501Y |
| P.1 | Brazil | December 2020 | E484K |
| (Gamma) | | | N501Y |
| B.1.617.2 | India | December 2020 | L452R |
| (Delta) | | | T478K |
| | | | 156del; 157 del |
| B.1.1.529 | South Africa | November 2021 | K417N, N440K, |
| (Omicron) | | | G446S, S477N, |
| | | | T478K, E484A, |
| | | | Q493K, G496S, |
| | | | Q498R, N501Y, |
| | | | Y505H and P681H. |

Thus, there is a need for prophylactic, therapeutic, and diagnostic agents useful against various variant strains of SARS-CoV-2, including variants that have not yet been identified and/or that have not yet emerged.

### SUMMARY

Described herein are highly conserved antigens and epitopes of SARS-CoV-2, binding proteins (e.g., antibodies) that bind to the disclosed antigens and epitopes, vaccines based on the antigens, methods of treating, preventing, or reducing the risks of infection with the antigens or binding proteins, and methods and kits for detecting or diagnosing infection by SARS-CoV-2 using the antigens or binding proteins.

In one aspect, the present disclosure provides isolated peptide antigens comprising or consisting of an amino acid sequence of any one of SEQ ID NOs: 2-25. In some embodiments, the isolated peptide antigen comprises or consists of an amino acid sequence of any one of SEQ ID NOs: 2-20. In some embodiments, the amino acid sequence is selected from any one of SEQ ID NOs: 3, 5, 8, 10-12 14, or 17-22. In some embodiments, the amino acid sequence is selected from any one of SEQ ID NOs: 18-20. In some embodiments, the isolated peptide antigen is recombinant.

In another aspect, the present disclosure provides vaccine compositions comprising an isolated peptide antigen comprising or consisting of an amino acid sequence of any one of SEQ ID NOs: 2-25 and a pharmaceutically acceptable carrier. In some embodiments, the isolated peptide antigen comprises or consists of an amino acid sequence of any one of SEQ ID NOs: 2-20. In some embodiments, the amino acid sequence is selected from any one or more of SEQ ID NOs: 3, 5, 8, 10-12, 14, or 17-22. In some embodiments, the amino acid sequence is selected from any one or more of SEQ ID NOs: 18-20. In some embodiments, the isolated peptide antigen is recombinant.

In another aspect, the present disclosure provides isolated binding proteins that bind to a conserved epitope of a coronavirus S protein, wherein the conserved epitope comprises the amino acid sequence of any one of SEQ ID NOs: 2-25. In some embodiments, the conserved epitope comprises or consists of an amino acid sequence of any one of SEQ ID NOs: 2-20. In some embodiments, the conserved epitope is selected from any one of SEQ ID NOs: 2-12 or 21-5. In some embodiments, the conserved epitope is selected from any one of SEQ ID NOs: 13-20. In some embodiments, the conserved epitope is selected from any one of SEQ ID NOs: 3, 5, 8, 10-12, 14, or 17-22. In some embodiments, the conserved epitope is selected from any one of SEQ ID NOs: 18-20. In some embodiments, the binding protein prevents the coronavirus S protein from binding to an angiotensin-converting enzyme 2 (ACE2) receptor. In some embodiments, the coronavirus is SARS-CoV-2 or a variant of SARS-CoV-2. In some embodiments, the isolated binding protein is an antibody or an antibody fragment, including a monoclonal antibody or fragment thereof.

In another aspect, the present disclosure provides methods of reducing the risk of a coronavirus infection in a subject, comprising administering to the subject an effective amount of a peptide antigen or a vaccine composition as disclosed herein. In another aspect, the present disclosure provides methods of treating, preventing, or reducing the risk of a coronavirus infection in a subject, comprising administering to the subject a therapeutically effective amount of a binding protein as disclosed herein. In some embodiments of the disclosed methods, the peptide antigen or binding protein is administered by subcutaneous or intramuscular injection. In some embodiments of the disclosed methods, the coronavirus is SARS-CoV-2 or a variant of SARS-CoV-2.

In another aspect, there is provided an in vitro method of analyzing a biological sample obtained from a subject, comprising contacting the sample with a binding protein that specifically binds to a SARS-CoV-2 peptide antigen selected from SEQ ID NOs: 2-25, and detecting binding between the binding protein and any SARS-CoV-2 antigen present in the sample. In some embodiments, the binding protein specifically binds to a SARS-CoV-2 peptide antigen selected from SEQ ID NOs: 2-20. In some embodiments, the method comprises contacting the sample with a panel of from 2 to 24 binding proteins that each specifically binds to a different SARS-CoV-2 peptide antigen selected from SEQ ID NOs: 2-25 or a panel of from 2 to 19 binding proteins that each specifically binds to a different SARS-CoV-2 peptide antigen selected from SEQ ID NOs: 2-20, and detecting binding between the binding proteins and any SARS-CoV-2 antigen present in the sample. In some embodiments, the sample is selected from saliva, nasal fluid, nasal cells, throat cells, blood, plasma, serum, urine, and feces. In some embodiments, the subject is suspected of having a SARS-CoV-2 infection, has been exposed to SARS-CoV-2, or is suspected of having been exposed to SARS-CoV-2. Some embodiments further comprise determining that the subject is infected with SARS-CoV-2 when binding is detected. In some embodiments, the sample comprises biological samples obtained from a plurality of subjects. Some such embodiments further comprise determining a level of infection in the plurality of subjects.

In another aspect, there is provided an in vitro method of analyzing a biological sample obtained from a subject, comprising contacting the sample with a SARS-CoV-2 peptide antigen comprising or consisting of an amino acid sequence selected from SEQ ID NOs: 2-25, and detecting binding between the peptide antigen and any anti-SARS-CoV-2 antibodies present in the sample. In some embodiments, the SARS-CoV-2 peptide antigen comprises or consists of an amino acid sequence selected from SEQ ID NOs: 2-20. In some embodiments, the method comprises contacting the sample with a panel of from 2 to 24 peptide antigens each comprising or consisting of a different an amino acid sequence selected from SEQ ID NOs: 2-25 or a panel of from 2 to 19 peptide antigens each comprising or consisting of a different an amino acid sequence selected from SEQ ID NOs: 2-20, and detecting binding between the peptide antigen and any anti-SARS-CoV-2 antibodies present in the sample. In some embodiments, the sample is selected from saliva, nasal fluid, nasal cells, throat cells, blood, plasma, serum, urine, and feces. In some embodiments, the subject is suspected of having a SARS-CoV-2 infection, has been exposed to SARS-CoV-2, or is suspected of having been exposed to SARS-CoV-2. Some embodiments further comprise determining that the subject is infected with SARS-CoV-2 when binding is detected. In some embodiments, the sample comprises biological samples obtained from a plurality of subjects. Some such embodiments further comprise determining a level of infection in the plurality of subjects.

In another aspect, the present disclosure also provides in vitro methods of analyzing a biological sample obtained from a subject, comprising extracting nucleic acids from the biological sample, contacting the extracted nucleic acids with a pair of primers that specifically amplify a nucleic acid sequence encoding a peptide of any one of SEQ ID NOs: 2-25, and detecting the presence of any amplified nucleic acid sequence present in the sample. In some embodiments the pair of primers specifically amplifies a nucleic acid sequence encoding a peptide of any one of SEQ ID NOs: 2-20. The methods may comprise contacting the sample with a panel of from 2 to 24 primer pairs each specific for a nucleic acid sequence that encodes a different amino acid sequence selected from SEQ ID NOs: 2-25 or a panel of from 2 to 19 primer pairs each specific for a nucleic acid sequence that encodes a different amino acid sequence selected from SEQ ID NOs: 2-20, and detecting amplification of each nucleic acid sequence if present in the sample. In some embodiments, the sample may be selected from saliva, nasal fluid, nasal cells, throat cells, blood, plasma, serum, urine, and feces. In some embodiments, the subject is suspected of having a SARS-CoV-2 infection, has been exposed to SARS-CoV-2, or is suspected of having been exposed to SARS-CoV-2. Some embodiments further comprise determining that the subject is infected with SARS-CoV-2 when amplification is detected. In some embodiments, the sample comprises biological samples obtained from a plurality of subjects. Some such embodiments further comprise determining a level of infection in the plurality of subjects.

In another aspect, the present disclosure provides kits comprising one or more binding proteins that each specifically binds to a different peptide comprising or consisting of any one of SEQ ID NOs: 2-25, a solid substrate to which the one or more binding proteins is attached, and a detectably labeled antibody that specifically binds to the peptide to which the one or more binding proteins specifically binds. In some embodiments, the one or more binding proteins each independently specifically bind to one or more peptides comprising or consisting of any one of SEQ ID NOs: 2-20. In another aspect, the present disclosure provides kits comprising one or more peptides each comprising or consisting of a different one of SEQ ID NOs: 2-25, a solid substrate to which the one or more peptides is attached, and a detectably labeled antibody that specifically binds to IgE or IgD, wherein the IgE or IgD are optionally human. In some embodiments, the one or more peptides comprise or consist of any one of SEQ ID NOs: 2-20. In some embodiments, the solid substrate is selected from a bead, a plate, a well, a dish, a slide, or a strip.

In another aspect, the present disclosure provides kits comprising one or more primer pairs each capable of specifically amplifying a nucleic acid sequence that encodes a different peptide selected from SEQ ID NOs: 2-25, wherein: (a) at least one primer of the primer pair is detectably labeled; or (b) the kit further comprises a detectably labeled probe that hybridizes to the nucleic acid sequence amplified by the primer pair. In some embodiments, the one or more primer pairs is each independently capable of specifically amplifying a nucleic acid sequence that encodes a different peptide selected from SEQ ID NOs: 2-20. In some embodiments, the kit comprises 1, 2, 3, 4, 5, 6, 7, 8, 9, 10, 11, 12, 13, 14, 15, 16, 17, 18, 19, 20, 21, 22, 23, or 24 primer pairs, each primer pair being capable of specifically amplifying a nucleic acid sequence that encodes a different peptide selected from SEQ ID NOs: 2-25 or SEQ ID NOs: 2-20.

In another aspect, the present disclosure provides isolated peptide antigens as disclosed herein for inducing an immune response to a coronavirus. In some embodiments, the peptide antigen is recombinant. In some embodiments, the coronavirus is SARS-CoV-2 or a variant of SARS-CoV-2.

In another aspect, the present disclosure provides isolated binding proteins as disclosed herein for treating, preventing, or reducing the risk of a coronavirus infection in a subject in need thereof. In some embodiments, the isolated binding protein is an antibody or an antibody fragment, including a monoclonal antibody or fragment thereof. In some embodiments, the coronavirus is SARS-CoV-2 or a variant of SARS-CoV-2.

In another aspect, the present disclosure provides uses of one or more isolated peptide antigens as disclosed herein in the preparation of a vaccine for inducing an immune response to a coronavirus. In some embodiments, the peptide antigen is recombinant. In some embodiments, the coronavirus is SARS-CoV-2 or a variant of SARS-CoV-2.

In another aspect, the present disclosure provides uses of one or more isolated binding proteins as disclosed herein in the preparation of a medicament for treating, preventing, or reducing the risk of a coronavirus infection in a subject in need thereof. In some embodiments, the isolated binding protein is an antibody or an antibody fragment, including a monoclonal antibody or fragment thereof. In some embodiments, the coronavirus is SARS-CoV-2 or a variant of SARS-CoV-2.

In another aspect, the present disclosure provides methods of preparing an antibody (such as a monoclonal antibody) that binds to a peptide antigen or epitope comprising or consisting of an amino acid sequence of any one of SEQ ID NOs: 2-25, including any one of SEQ ID NOs: 2-20, comprising: (a) identifying an asymptomatic patient that has been infected with a coronavirus as a donor for obtaining immune B-lymphocytes that produce high titers of coronavirus-neutralizing antibodies; (b) collecting the B-lymphocytes from the patient; (c) immortalizing the B-lymphocytes; (d) collecting antibodies produced by the immortalized B-lymphocytes; and (e) screening the antibodies for binding to a peptide antigen or epitope comprising or consisting of an amino acid sequence of any one of SEQ ID NOs: 2-25 (or any one of SEQ ID NOs: 2-20). In some embodiments, the method may further comprise testing the antibodies for binding to SARS-CoV-2. In some embodiments, the method may further comprise epitope mapping the antibodies that tested positive for binding to SARS-CoV-2. In some embodiments, immortalizing the B-lymphocytes comprises fusing a B-lymphocyte with a heteromyeloma cell in order to produce a heterohybridoma cell.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIG. 1 shows an alignment of the furin cleavage site of several isolates of SARS-CoV-2.

### DETAILED DESCRIPTION

The present disclosure provides highly conserved antigens and epitopes of the SARS-CoV-2 S protein (i.e., spike protein). The disclosed antigens and epitopes are highly conserved across all viral isolates, indicating that there is strong selective pressure to maintain these sequences, even as variants of SARS-CoV-2 develop. As a result, proteins (e.g. antibodies) that bind to the disclosed antigens and epitopes may provide pan-protection, both in terms of treatment and prevention, against variant strains of SARS-CoV-2, and potentially other β-coronaviruses as well. Similarly, vaccines based on the disclosed highly conserved antigens may provide broadly universal and temporally durable protection against a range of SARS-CoV-2 variants and coronavirus strains. Also, diagnostic methods and kits based on the disclosed antigens may permit detection of infection by known and future strains of SARS-CoV-2.

### I. Definitions

It is to be understood that the terminology used herein is for the purpose of describing particular embodiments only, and is not intended to be limiting.

Technical and scientific terms used herein have the meanings commonly understood by one of ordinary skill in the art, unless otherwise defined. Unless otherwise specified, materials and/or methodologies known to those of ordinary skill in the art can be utilized in carrying out the methods described herein, based on the guidance provided herein.

As used herein, the singular terms “a,” “an,” and “the” include plural referents unless the context clearly dictates otherwise. Reference to an object in the singular is not intended to mean “one and only one” unless explicitly so stated, but rather “one or more.”

As used herein, “about” when used with a numerical value means the numerical value stated as well as plus or minus 10% of the numerical value. For example, “about 10” should be understood as both “10” and “9-11.”

As used herein, a phrase in the form “A/B” or in the form “A and/or B” means (A), (B), or (A and B); a phrase in the form “at least one of A, B, and C” means (A), (B), (C), (A and B), (A and C), (B and C), or (A, B, and C).

As used herein, the term “comprising” is intended to mean that the compositions and methods include the recited elements, but does not exclude others.

An used herein, the term “isolated” when used in the context of referring to a peptide antigen or binding protein or antibody as discussed herein refers to one which has been separated from at least some of the components with which it existed in nature (for those isolated from nature) or with which it was produced (for those produced, e.g., in a laboratory setting).

As used herein, a “variant” when used in the context of referring to a peptide means a peptide sequence that is derived from a parent sequence by incorporating one or more amino acid changes, which can include substitutions, deletions, or insertions. For the purposes of this disclosure, a variant may comprise an amino acid sequence that shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or up to about 100% sequence identity or homology with the reference (or “parent”) sequence. For purposes of this disclosure, the terms “variant” and “derivative” when used in the context of referring to a peptide are used interchangeably.

As used herein, the phrases “effective amount,” “therapeutically effective amount,” and “therapeutic level” mean the dosage or concentration of an antigen, antibody or binding protein that provides the specific pharmacological effect for which the antigen, antibody or binding protein is administered in a subject in need of such treatment, e.g., to induce a protective immune response against coronavirus or to treat or prevent a coronavirus infection (e.g., MERS, SARS, or COVID-19). It is emphasized that a therapeutically effective amount or therapeutic level of an antigen, antibody or binding protein will not always be effective in inducing a protective immune response or treating or preventing the infections described herein, even though such dosage is deemed to be a therapeutically effective amount by those of skill in the art. The therapeutically effective amount may vary based on the route of administration and dosage form, the age and weight of the subject, and/or the subject's condition, including the type and severity of the coronavirus infection.

The terms “treat,” “treatment” or “treating” as used herein with reference to a coronavirus infection refer to reducing or eliminating viral load.

The terms “prevent,” “preventing” or “prevention” as used herein with reference to a coronavirus infections refer to precluding or reducing the risk of an infection from developing in a subject exposed to a coronavirus, or to precluding or reducing the risk of developing a high viral load of coronavirus. Prevention may also refer to the prevention of a subsequent infection once an initial infection has been treated or cured.

The terms “individual,” “subject,” and “patient” are used interchangeably herein, and refer to any individual mammalian subject, e.g., bovine, canine, feline, equine, or human. In specific embodiments, the subject, individual, or patient is a human.

### II. SARS-CoV-2 and S Protein

SARS-CoV-2 is a single-stranded RNA-enveloped virus. An RNA-based metagenomic next-generation sequencing approach has been applied to characterize its entire genome, which is 29,881 bp in length (GenBank no. MN908947), encoding 9860 amino acids. The genome of SARS-CoV-2 comprises 13-15 reading frames (ORFs) containing ˜30,000 nucleotides and of which 12 are functional. The ORFs are arranged as replicase and protease, and are major drug/vaccine targets. The whole genome of SARS-CoV-2 encodes a polyprotein that is about 7,096 residues long and which comprises both structural and non-structural proteins. The four main structural proteins include spike (S), membrane (M), envelope (E), and nucleocapsid (N) proteins, and are encoded by ORFs 10,11 on the third of the genome near the 3′-terminus.

Glycosylated S proteins (also known as a “spike protein”) cover the surface of SARS-CoV-2 and bind to the host cell receptor angiotensin-converting enzyme 2 (ACE2), mediating viral cell entry. When the S protein binds to the receptor, TM protease serine 2 (TMPRSS2), a type 2 TM serine protease located on the host cell membrane, promotes virus entry into the cell by activating the S protein. Once the virus enters the cell, the viral RNA is released, polyproteins are translated from the RNA genome, and replication and transcription of the viral RNA genome occur via protein cleavage and assembly of the replicase—transcriptase complex. Viral RNA is replicated, and structural proteins are synthesized, assembled, and packaged in the host cell, after which viral particles are released.

The SARS-CoV-2 S protein is highly conserved among all human coronaviruses (HCoVs) and is involved in receptor recognition, viral attachment, and entry into host cells. Due to its indispensable functions, it represents one of the most important targets for COVID-19 treatment and prevention.

The S protein of SARS-CoV-2 is 180-200 kDa and spans 1273 amino acids (NCBI Reference Sequence: YP_009724390.1). The structure includes an extracellular N-terminus, a transmembrane (TM) domain anchored in the viral membrane, and a short intracellular C-terminal segment. S normally exists in a metastable, prefusion conformation; once the virus interacts with the host cell, extensive structural rearrangement of the S protein occurs, allowing the virus to fuse with the host cell membrane. The amino acid sequence (SEQ ID NO:1) is shown below:

MFVFLVLLPLVSSQCVNLTTRTQLPPAYTNSFTRGVYYPDKVFRSSVL
HSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVY
YHKNNKSWMESEFRVYSSANNCTFEYVSQPFLMDLEGKQGNFKNLREF
VFKNIDGYFKIYSKHTPINLVRDLPQGFSALEPLVDLPIGINITRFQT
LLALHRSYLTPGDSSSGWTAGAAAYYVGYLQPRTFLLKYNENGTITDA
VDCALDPLSETKCTLKSFTVEKGIYQTSNFRVQPTESIVRFPNITNLC
PFGEVENATRFASVYAWNRKRISNCVADYSVLYNSASFSTFKCYGVSP
TKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPK
KSTNLVKNKCVNFNFNGLTGTGVLTESNKKFLPFQQFGRDIADTTDAV
RDPQTLEILDITPCSFGGVSVITPGTNTSNQVAVLYQDVNCTEVPVAI
HADQLTPTWRVYSTGSNVFQTRAGCLIGAEHVNNSYECDIPIGAGICA
SYQTQTNSPRRARSVASQSIIAYTMSLGAENSVAYSNNSIAIPTNFTI
SVTTEILPVSMTKTSVDCTMYICGDSTECSNLLLQYGSFCTQLNRALT
GIAVEQDKNTQEVFAQVKQIYKTPPIKDFGGFNFSQILPDPSKPSKRS
FIEDLLFNKVTLADAGFIKQYGDCLGDIAARDLICAQKFNGLTVLPPL
LTDEMIAQYTSALLAGTITSGWTFGAGAALQIPFAMQMAYRFNGIGVT
QNVLYENQKLIANQFNSAIGKIQDSLSSTASALGKLQDVVNQNAQALN
TLVKQLSSNFGAISSVLNDILSRLDKVEAEVQIDRLITGRLQSLQTYV
TQQLIRAAEIRASANLAATKMSECVLGQSKRVDFCGKGYHLMSFPQSA
PHGVVFLHVTYVPAQEKNFTTAPAICHDGKAHFPREGVFVSNGTHWFV
TQRNFYEPQIITTDNTFVSGNCDVVIGIVNNTVYDPLQPELDSFKEEL
DKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCC
SCGSCCKFDEDDSEPVLKGVKLHYT

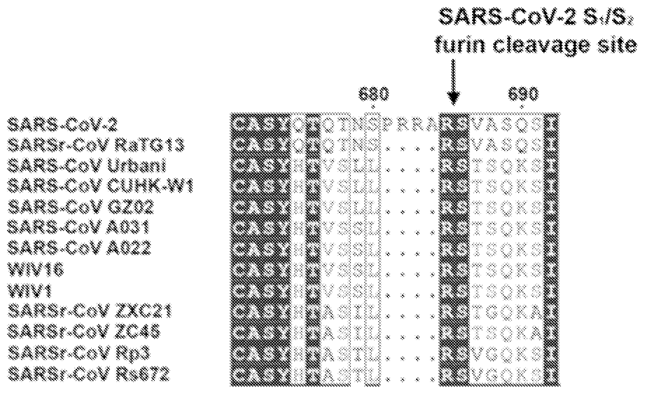

The S protein comprises two subunits: the S1 subunit and the S2 subunit. The S1 subunit is involved in the attachment of virions with the host cell membrane by interacting with human ACE2 that subsequently initiates the infection process. The S1 subunit comprises amino acid residues 1-685 of the S protein of SEQ ID NO:1, with amino acids 1-13 serving as a signal peptide at the N terminus. Within the S1 subunit there is a receptor binding domain (RBD), which comprises amino acid residues 319-591 of SEQ ID NO:l. The other subunit of the S protein, S2 works as the fusion protein that helps in the fusion of the virion with the cell membrane. The S2 subunit comprises amino acid residues 686-1273 of the S protein of SEQ ID NO:1. Furin protease cleavage between residues 685 and 686 separates the S1 and S2 subunits. The furin cleavage site of several isolates of SARS-CoV-2 is shown in FIG. 1.

The S2 subunit normally contains multiple key components, including one or more fusion peptides (FP), a second proteolytic site (S2′) and two conserved heptad repeats (HRs), driving membrane penetration and virus-cell fusion. The HRs can trimerize into a coiled-coil structure built of three HR1-HR2 helical hairpins presenting as a canonical six-helix bundle and drag the virus envelope and the host cell bilayer into close proximity, preparing for fusion to occur. The fusion core is composed of HR1 and HR2 and at least three membranotropic regions that are denoted as the fusion peptide (FP), internal fusion peptide (IFP), and pretransmembrane domain (PTM). The HR regions are further flanked by the three membranotropic components. Both FP and IFP are located upstream of HR1, while PTM is distally downstream of HR2 and directly precedes the transmembrane domain of SARS-CoV-2. All of these three components are able to partition into the phospholipid bilayer to disturb membrane integrity.

### III. Antigens and Epitopes of the S Protein

The present disclosure provides highly conserved peptide sequences of the SARS-CoV-2 S protein that can be used as antigens or epitopes of binding proteins (e.g., antibodies) for vaccines, targets for drugs and/or for treating and/or preventing and/or diagnosing SARS-CoV-2 infection.

More specifically, the disclosed epitopes of SEQ ID NOs: 2-20 were discovered using computational prediction and machine learning approaches to assess and compare 50,512 viral isolates to identify highly conserved regions. A further analysis of 57,000 SARS-CoV-2 viral isolates showed high conservation of the disclosed epitopes of SEQ ID NOs: 2-20 across the analyzed isolates, including being conserved in the original Wuhan strain and the Alpha (B.1.1.7), Beta (B.1.351), and Gamma variants. A further analysis of 2 million SARS-CoV-2 viral isolates showed high conservation of the disclosed epitopes of SEQ ID NOs: 2-20 across the analyzed isolates, including being conserved in the original Wuhan strain and the Alpha (B.1.1.7), Beta (B.1.351), Gamma (P.1), Delta (B.1.617.2), Lambda (C.7) and Mu (B.1.621) variants. A further analysis of 2 million SARS-CoV-2 viral isolates showed high conservation of the disclosed epitopes of SEQ ID NOs: 2-20 across the analyzed isolates, including being conserved in the original Wuhan strain and the Alpha (B.1.1.7), Beta (B.1.351), Gamma (P.1), Delta (B.1.617.2), Lambda (C.7), Mu (B.1.621), and Omicron (B.1.1.529) variants. The disclosed epitopes of SEQ ID NOs: 21-25 were discovered in a further analysis of 2.8 million SARS-CoV-2 viral isolates analyzed for epitopes conserved in the Delta and Omicron variants.

The pronounced conservation of these regions across this many isolates indicates that these regions are unlikely to mutate as SARS-CoV-2 evolves and spawns new variants. The conserved nature of these epitopes also indicates that antibodies or other binding proteins that bind to these regions and vaccines that contain an antigen that comprises or consists of a peptide having one of the amino acid sequences may provide broad treatment and/or protection against a wide range of coronaviruses, particularly β-coronaviruses, and that these epitopes and antibodies or other binding proteins that bind to these regions can be used in diagnostic assays useful for detecting or diagnosing SARS-CoV-2 infection on an individual or community level.

Specific antigens/epitopes of the S2 subunit of the SARS-CoV-2 spike protein disclosed herein are shown in Table 1 below, along with the percent conservation of each epitope across the 50,512 viral isolates of the initial analysis (for SEQ ID NOs: 2-20), or across 2.8 million isolates from Delta and Omicron variants (for SEQ ID NOs: 21-25).

TABLE 1
S2 Subunit Antigens/Epitopes

| | | Residues | | SEQ |
|---|---|---|---|---|
| | | in S | Percent | ID |
| Name | Sequence | Protein | Conserved | NO: |
| S2-Seq-1 | NGTHWF | 1098-1103 | 99.285%* | 2 |
| S2-Seq-2 | CDVVIGIVN | 1126-1134 | 99.260%* | 3 |
| S2-Seq-3 | TSGWTFG | 883-889 | 99.222%* | 4 |
| S2-Seq-4 | RDLICAQKFN | 847-856 | 99.180%* | 5 |
| S2-Seq-5 | NQKLIAN | 919-925 | 99.135%* | 6 |
| S2-Seq-6 | TASALGKL | 941-948 | 99.133%* | 7 |
| S2-Seq-7 | CLKGCC | 1243-1248 | 99.093%* | 8 |
| S2-Seq-8 | NGIGVTQN | 907-914 | 99.066%* | 9 |
| S2-Seq-9 | CTMYICGDST | 738-750 | 98.844%* | 10 |
| | ECS | | | |
| S2-Seq-10 | CTMYICGDST | 738-760 | 98.262%* | 11 |
| | ECSNLLLQYG | | | |
| | SFC | | | |
| S2-Seq-11 | NLLLQYGSFC | 751-760 | 97.680%* | 12 |
| S2-Seq-12 | CVLGQSKRVD | 1032-1044 | 96.283%** | 21 |
| | FCG | | | |
| S2-Seq-13 | NFTTAPAICH | 1074-1083 | 94.901%** | 22 |
| S2-Seq-14 | KPSKRSFIED | 811-824 | 95.570%** | 23 |
| | LLFN | | | |
| S2-Seq-15 | KYEQYIKW | 1205-1212 | 95.990%** | 24 |
| S2-Seq-16 | FIEDLLFN | 817-824 | 96.648%** | 25 |

*Percent conserved across 50,512 viral isolates.
**Percent conserved across 2.8 million isolates from Delta and Omicron variants

Specific antigens/epitopes of the S1 subunit of the SARS-CoV-2 spike protein disclosed herein are shown in Table 1 below, along with the percent conservation of each epitope across the 50,512 viral isolates of the initial analysis.

TABLE 2
S1 Subunit Antigens/Epitopes

| | | Residues | | SEQ |
|---|---|---|---|---|
| | | in S | Percent | ID |
| Name | Sequence | Protein | Conserved* | NO: |
| S1-Seq-1 | VYYPDKVFRSS | 36-51 | 98.523% | 13 |
| | VLHST | | | |
| S1-Seq-2 | KVCEFQFCNDP | 129-146 | 96.553% | 14 |
| | FLGVYYH | | | |
| S1-Seq-3 | LKSFTVEKGIY | 300-314 | 90.375% | 15 |
| | QTSN | | | |
| S1-Seq-4 | YNENGTITDAV | 276-287 | 90.612% | 16 |
| | D | | | |
| S1-Seq-5 | CPFGEVFNATR | 333-348 | 98.668% | 17 |
| | FASVYAWN | | | |
| S1-Seq-6 | LCFTNVYADSF | 386-406 | 97.078% | 18 |
| | VIRGDEVRQ | | | |
| S1-Seq-7 | KLPDDFTGCVI | 421-437 | 98.705% | 19 |
| | AWNSNN | | | |
| S1-Seq-8 | PYRVVVLSFEL | 506-527 | 91.258% | 20 |
| | LHAPATVCGPK | | | |

*Percent conserved across 50,512 viral isolates.

The percent conservation results of the other isolate analyses of SEQ ID NOs: 2-20 are shown below in Table 3. In particular, this table shows the percent conservation of SEQ ID NOs: 2-20 across 57,500 isolates of the original Wuhan strain and the Alpha, Beta, and Gamma variants (first column of values); across 2 million isolates of the original Wuhan strain and the Alpha, Beta, Gamma, Delta, Lambda, and Mu variants (second column of values); and across 2 million isolates of the original Wuhan strain and the Alpha, Beta, Gamma, Delta, Lambda, Mu, and Omicron variants (third column of values). Thus, for example, SEQ ID NO:13 was observed to have 100% conservation across 2 million isolates of the original Wuhan strain and Alpha, Beta, Gamma, Delta, Lambda, Mu, and Omicron variants (third column). In contrast, SEQ ID NO:14 was observed to be greater than 96% conserved in the original Wuhan strain (Table 2), but was poorly conserved across the subsequently identified variants (Table 3). Both sequences may nevertheless be useful. For instance, whereas SEQ ID NO:13 could be used for a broad spectrum antigen (such as for a vaccine), an epitope target for a broad spectrum antibody (such as for passive immunotherapy), or a good target sequence for a broad spectrum diagnostic, SEQ ID NO:14 would be well suited for isolation, selective diagnosis, or selective treatment of the original Wuhan strain.

TABLE 3
Additional Isolate Conservation Studies

| | 57,500 | 2 Million | 2 Million | |
|---|---|---|---|---|
| | SARS-CoV-2 | SARS-CoV-2 | SARS-CoV-2 | |
| | ISOLATES | ISOLATES | ISOLATES | |
| | Including | Including | Including Wuhan | |
| | Wuhan strain | Wuhan strain | strain and Alpha, | |
| | and Alpha, | and Alpha, Beta, | Beta, Gamma, | |
| | Beta and | Gamma, Delta, | Delta, Lambda, | |
| | Gamma | Lambda, and Mu | Mu and Omicron | SEQ |
| Epitope Sequence | variants | variants | variants | ID NO: |
| VYYPDKVFRSSVLHST | 99.4% | 99% | 100% | 13 |
| KVCEFQFCNDPFLGVYYH | 34.0% | 3.1% | 0% | 14 |
| LKSFTVEKGIYQTSN | 96.3% | 93.8% | 100% | 15 |
| YNENGTITDAVD | 96.1% | 94.5% | 100% | 16 |
| CPFGEVFNATRFASVYAWN | 97.7% | 0.3% | 0% | 17 |
| LCFTNVYADSFVIRGDEVRQ | 98.6% | 95.9% | 100% | 18 |
| KLPDDFTGCVIAWNSNN | 98.3% | 95.5% | 10% | 19 |
| PYRVVVLSFELLHAPATVCGPK | 97.7% | 94.5% | 100% | 20 |
| NGTHWF | 97.7% | 95.2% | 100% | 2 |
| CDVVIGIVN | 98.1% | 95.5% | 100% | 3 |
| TSGWTFG | 98.6% | 95.5% | 100% | 4 |
| RDLICAQKFN | 97.9% | 95.9% | 10% | 5 |
| NQKLIAN | 98.4% | 95.9% | 100% | 6 |
| TASALGKL | 98.1% | 95.9% | 100% | 7 |
| CLKGCC | 97.6% | 95.2% | 100% | 8 |
| NGIGVTQN | 98.6% | 95.5% | 100% | 9 |
| CTMYICGDSTECS | 98.4% | 94.9% | 100% | 10 |
| CTMYICGDSTECSNLLLQYGSFC | 98.4% | 94.5% | 100% | 11 |
| NLLLQYGSFC | 98.7% | 95.5% | 100% | 12 |

Three-dimensional artificial intelligence analysis of the SARS-CoV-2 spike protein was undertaken to identify accessible regions for antibody binding, and it was determined that all of the disclosed epitopes are linear epitopes on the spike protein. In particular, starting with the SARS-CoV-2 spike ectodomain structure provided by the RCSB Protein Data Bank (available at rcsb.org/structure/6VYB), a three-dimensional model for the hetero-trimeric spike protein was constructed. Using this model, individual models were built for each of the 24 epitopes disclosed herein. These models indicate the epitopes are conserved, accessible, and linear on the spike protein.

The peptide sequences disclosed in Tables 1 and 2 can be used as antigens in a vaccine or can be used to develop a binding protein, such as an antibody, to be used in passive immunization or therapy. Any of the peptide sequences in Tables 1 or 2 (i.e., SEQ ID NOs: 2-25) can be formulated in a vaccine to immunize a subject and/or to induce an immune response (e.g., to induce antibodies) in the subject, and can be used singly or in combination (e.g., in multivalent vaccines or multivalent diagnostics as discussed below). Alternatively, any of the peptide sequences in Tables 1 or 2 (i.e., SEQ ID NOs: 2-25) can be used to produce (raise) antibodies, such as by immunizing an animal, such as a mouse or rat or human, to produce (raise) antibodies that specifically bind to the immunizing peptide sequence, including to produce monoclonal antibodies. The antibodies induced or produced by such methods will bind to the corresponding sequence on the S protein, e.g., will bind to that epitope on the S protein. Such antibodies can be used singly or in combination for analytical purposes or in passive immunotherapy as discussed below.

Studies indicate that the antigens/epitopes comprising a cysteine residue may be the most immunogenic and likely to give rise to neutralizing antibodies. Thus, peptides comprising or consisting of any of SEQ ID NOs: 3, 5, 8, 10-12, 14, or 17-22 may be particularly useful as vaccine antigens, and antibodies raised against peptides comprising or consisting of any of SEQ ID NOs: 3, 5, 8, 10-12, 14, or 17-22 may be particularly useful for passive immunization or passive immunotherapy therapy. Further studies indicate that SEQ ID Nos: 18-20 are likely to give rise to neutralizing antibodies. Thus, peptides comprising or consisting of any of SEQ ID NOs: 18-20 may be particularly useful as vaccine antigens, and antibodies raised against peptides comprising or consisting of any of SEQ ID NOs: 18-20 may be particularly useful for passive immunization or passive immunotherapy therapy.

### IV. Coronavirus Vaccines

The present disclosure provides vaccines comprising one or more antigen(s) that comprise or consist of a peptide having one of the amino acid sequences in Tables 1 or 2 (i.e., SEQ ID NOs: 2-25). In some embodiments, the vaccines comprise one or more antigen(s) that comprise or consist of a peptide having an amino acid sequence of any of SEQ ID NOs: 2-20. In some embodiments, the vaccines comprise one or more antigen(s) that comprise or consist of a peptide having an amino acid sequence of any of SEQ ID NOs: 3, 5, 8, 10-12, 14, or 17-22, including one or more of SEQ ID Nos:18-20. The antigens can be prepared by methods known in the art, such as chemical synthesis, or by recombinant methods. Techniques for making peptides are known in the art, and can be used to obtain antigens as disclosed herein. The vaccines may comprise one or more antigen(s) formulated in a pharmaceutically acceptable carrier for the intended route of administration, as discussed in more detail below.

The immune response elicited by immunization with a vaccine as disclosed herein (e.g., comprising one or more antigen(s) that comprise or consist of a peptide having one of the sequences in Tables 1 or 2 (SEQ ID NOs: 2-25)) is expected to induce production of antibodies that bind highly conserved epitopes of SARS-CoV-2 and provide broad spectrum immune protection against SARS-CoV-2 and variants thereof. The antigenic sequences disclosed in Table 1 (SEQ ID NOs: 2-12 and 21-25) are particularly unique because development and production of vaccines against SARS-CoV-2 has, to date, focused almost exclusively on the S1 subunit of the S protein.

A vaccine as disclosed herein, comprising one or more antigen(s) that comprise or consist of one or more of the peptide sequences in Tables 1 or 2 (i.e., each comprising or consisting of one of SEQ ID NOs: 2-25), can be used for treating or preventing a coronavirus infection, particularly a β-coronavirus infection such as a SARS-CoV-2 infection (e.g., COVID-19). Optimal doses and routes of administration may vary, such as based on the route of administration and dosage form, the age and weight of the subject, and/or the subject's condition, and can be determined by the skilled practitioner. The vaccine may be formulated for injection and administered parenterally, such as intramuscularly, subcutaneously, or intradermally. The vaccine may be formulated for intravenous injection or infusion. The disclosed vaccines may be formulated to be administered alone or concurrently with another therapeutic agent for treating a coronavirus infection. The vaccines may be formulated to be administered in sequence with another therapeutic agent. For example, the vaccine may be administered either before or after the subject has received a regimen of an anti-viral therapy. The vaccines may be administered as a single dose or an initial dose followed by one or more booster doses.

A vaccine as disclosed herein, comprising one or more antigen(s) that comprise or consist of one or more of the peptide sequences in Tables 1 or 2 (i.e., each comprising or consisting of one of SEQ ID NOs: 2-25), can be formulated or administered with an adjuvant to improve immune responses and promote protective responses. An adjuvant is an ingredient used in some vaccines that helps create a stronger immune response in people receiving the vaccine. Adjuvants help the body to produce an immune response strong enough to protect the person from the disease he or she is being vaccinated against. Those skilled in the art are aware of pharmaceutically acceptable adjuvants that may be combined with one or more of the disclosed antigens to prepare a vaccine.

### V. Coronavirus Binding Proteins

The present disclosure also provides binding proteins, such as antibodies, including monoclonal antibodies, that specifically bind to the disclosed antigen/epitope sequences (i.e., to one of SEQ ID NOs: 2-25). In some embodiments, the binding proteins may selectively bind to an epitope that comprises or consists of a peptide having an amino acid sequence of any of SEQ ID NOs: 2-25. In some embodiments, the binding proteins may selectively bind to an epitope that comprises or consists of a peptide having an amino acid sequence of any of SEQ ID NOs: 2-20. In some embodiments, the binding proteins may selectively bind to an epitope that comprises or consists of a peptide having an amino acid sequence of any of SEQ ID NOs: 3, 5, 8, 10-12, 14, or 17-22, including one or more of SEQ ID Nos:18-20. While not wanting to be bound by theory, the disclosed binding proteins can prevent a coronavirus from binding an ACE2 receptor and propagating an infection. The disclosed binding proteins can be used for passive immunization or as therapeutics or diagnostics. The disclosed binding proteins can be used in methods for treating, preventing, or reducing the risk of coronavirus infection (e.g., COVID-19) or the development of a coronavirus infection in an individual in need thereof. For example, the disclosed binding proteins can be administered in a therapeutically effective amount to a subject in need thereof to reduce circulating levels of coronavirus (such as SARS-CoV-2), reduce viral load, and/or reduce, ameliorate, or eliminate one or more signs or symptoms of coronavirus infection.

The disclosed binding proteins include antibodies and antibody fragments, monomers, dimers, single-domain antibodies, and other immunoglobulin fragments, variants, or derivatives. The binding proteins disclosed herein can be obtained by any means, including from in vitro sources (e.g., a hybridoma or a cell line producing the peptide recombinantly) and in vivo sources (e.g., rodents, rabbits, humans, etc.). In some embodiments, the binding proteins may be produced by a heterohybridoma, as discussed in more detail below. In some embodiments, the binding proteins may be monoclonal antibodies.

The binding proteins disclosed herein specifically bind to an epitope on the SARS-CoV-2 S protein disclosed herein (i.e., to one of SEQ ID NOs: 2-25). In some embodiments, one of SEQ ID NOs: 2-25 may represent the minimal epitope to which the disclosed binding proteins specifically bind, i.e., the minimal essential core epitope(s).

In general, the disclosed binding proteins comprise at least a least a portion of an immunoglobulin heavy chain. For instance, in some embodiments, the binding protein may comprise a heavy chain monomer, a heavy chain dimer, or may be a single-domain antibody (i.e., a V_{H}H fragment, a “nanobody,” or a “camelid-like” antibody). A single-domain antibody may comprise or consist of a V_{H} domain, a C_{H2} domain, and a C_{H3} domain, but not a V_{K} domain or a C_{H1} domain.

The disclosed binding proteins can comprise, but do not require, an immunoglobulin light chain in order to bind a coronavirus S protein epitope disclosed herein. In some embodiments, the disclosed binding proteins comprise both a heavy and light chain. In some embodiments, the disclosed binding proteins are full antibodies (e.g., complete IgGs). Human, partially humanized, fully humanized, and chimeric versions of the binding protein disclosed can be made by methods known in the art, such as using a transgenic animal (e.g., a mouse) wherein one or more endogenous immunoglobulin gene sequences are replaced with one or more human immunoglobulin gene sequences. Examples of transgenic mice wherein endogenous antibody genes are effectively replaced with human antibody genes include, but are not limited to, the HUMAB-MOUSE™, the Kirin TC MOUSE™, and the KM-MOUSE™ (see, e.g., Lonberg, Nat. Biotechnol., 23(9): 1117-25 (2005), and Lonberg, Handb. Exp. Pharmacol., 181: 69-97 (2008)).

The disclosed binding proteins may be an antibody. Typically, an antibody consists of four polypeptides: two identical copies of a heavy (H) chain polypeptide and two copies of a light (L) chain polypeptide. Each heavy chain contains one N-terminal variable (V_{H}) region and three C-terminal constant (C_{H}1, C_{H}2 and C_{H}3) regions, and each light chain contains one N-terminal variable (V_{L}) region and one C-terminal constant (C_{L}) region. The variable regions of each pair of light and heavy chains form the antigen binding site of an antibody, however, some of the disclosed peptides may comprise a heavy chain without a light chain. Light and heavy chain variable regions contain a “framework” region interrupted by three hypervariable regions, also called “complementarity-determining regions” or “CDRs.” The extent of the framework region and CDRs has been defined (see Kabat et al., Sequences of Proteins of Immunological Interest, U.S. Department of Health and Human Services, 1991). The Kabat database is now maintained online. The sequences of the framework regions of different light or heavy chains are relatively conserved within a species, and framework regions act to form a scaffold that provides for positioning the CDRs in correct orientation by inter-chain, non-covalent interactions.

The disclosed binding proteins may be an “antibody fragment,” which refers to one or more portions of a coronavirus-binding antibody that exhibits the ability to bind to an epitope on the SARS-CoV-2 S protein defined by any one of SEQ ID NOs: 2-25. Examples of binding fragments include (i) Fab fragments (monovalent fragments consisting of the V_{L}, V_{H}, C_{L} and C_{H1} domains); (ii) F(ab′)₂ fragments (bivalent fragment comprising two Fab fragments linked by a disulfide bridge at the hinge region); (iii) Fd fragments (comprising the V_{H} and C_{H1} domains); (iv) Fv fragments (comprising the V_{L} and V_{H} domains of a single arm of an antibody), (v) dAb fragments (comprising a V_{H} domain); and (vi) isolated complementarity determining regions (CDRs), e.g., V_{H}CDR3. Other examples include single chain Fv (scFv) constructs. See e.g., Bird et al., Science, 242:423-26 (1988); Huston et al., Proc. Natl. Acad. Sci. USA, 85:5879-83 (1988). Other examples of types of antibody fragments include coronavirus-binding domain immunoglobulin fusion proteins comprising (i) a coronavirus-binding domain polypeptide (such as a heavy chain variable region, a light chain variable region, or a heavy chain variable region fused to a light chain variable region via a linker peptide) fused to an immunoglobulin hinge region polypeptide, (ii) an immunoglobulin heavy chain C_{H2} constant region fused to the hinge region, and (iii) an immunoglobulin heavy chain C_{H3} constant region fused to the C_{H2} constant region, where the hinge region may be modified by replacing one or more cysteine residues with, for example, serine residues, to prevent dimerization.

As noted above, the disclosed binding proteins may or may not comprise a light chain. Similarly, he disclosed binding proteins may or may not comprise a CH1 region. For instance, in some embodiments, a binding protein may comprise or consist of a V_{H} domain, a C_{H2} domain, and a C_{H3} domain. In some embodiments, a binding protein may comprise or consist of a V_{H} domain, a C_{H1} domain, a C_{H2} domain, and a C_{H3} domain. In some embodiments, the constant domains may comprise one or more modifications, such as an amino acid substitution.

The disclosed binding proteins include monoclonal antibodies (mAbs) and fragments thereof, which may be obtained by methods known in the art, for example, by fusing antibody-producing cells with immortalized cells to obtain a hybridoma, and/or by generating mAbs from mRNA extracted from bone marrow, B cells, and/or spleen cells of immunized animals using combinatorial antibody library technology and/or by isolating monoclonal antibodies from serum from subjects immunized with a peptide antigen, such as a peptide antigen comprising or consisting of any one of SEQ ID NOs: 2-25, including any one of SEQ ID NOs: 2-20, or a peptide antigen comprising or consisting of any of SEQ ID NOs: 3, 5, 8, 10-12, 14, or 17-22, including one or more of SEQ ID Nos:18-20; or created from “immune B-cells” obtained from convalescent COVID-19 patients' peripheral mononuclear cells.

Recombinant versions of the disclosed binding proteins may be obtained by methods known in the art, for example, using phage display technologies, yeast surface display technologies (Chao et al., Nat. Protoc., 1(2): 755-68 (2006)), mammalian cell surface display technologies (Beerli et al., PNAS, 105(38): 14336-41 (2008), and/or by expressing or co-expressing component polypeptides, such as heavy and light chain polypeptides. Other techniques for making peptides and antibodies are known in the art, and can be used to obtain binding proteins as well.

The disclosed binding proteins may be or be derived from a human IgG1 antibody, a human IgG2 antibody, a human IgG3 antibody, or a human IgG4 antibody. In some embodiments, the binding protein may be or be derived from a class of antibody selected from IgG, IgM, IgA, IgE, and IgD. That is, the disclosed binding proteins may comprise all or part of the constant regions, framework regions, or a combination thereof of an IgG, IgM, IgA, IgE, or IgD antibody. For instance, a disclosed binding protein comprising an IgG1 immunoglobulin structure may be modified to replace (or “switch”) the IgG1 structure with the corresponding structure of another IgG-class immunoglobulin or an IgM, IgA, IgE, or IgD immunoglobulin. This type of modification or switching may be performed in order to augment the neutralization functions of the peptide, such as antibody dependent cell cytotoxicity (ADCC) and complement fixation (CDC). A person of ordinary skill in the art will understand that, for example, a recombinant IgG1 immunoglobulin structure can be “switched” to the corresponding regions of immunoglobulin structures from other immunoglobulin classes, such as recombinant secretory IgA1 or recombinant secretory IgA2, such as may be useful for topical application onto mucosal surfaces. For example, immunoglobulin IgA structures are known to have applications in protective immune surveillance directed against invasion of infectious diseases, which makes such structures suitable for methods of using the disclosed binding proteins in such contexts, e.g., treating or preventing coronavirus infection (e.g., COVID-19) or the spread of coronavirus from one individual to another.

In some embodiments, a disclosed binding protein may comprise one or more mutations, alterations, or modifications that improve one or more properties or functions of the binding protein. Such mutations, alterations, or modifications may comprise, for example, changes to the Fc region to increase the ability of the peptide to mediate cellular cytotoxicity functions like antibody dependent cell cytotoxicity (ADCC), antibody dependent cell mediated phagocytosis (ADCP), and/or complement fixation (CDC). A wide number of mutations to the Fc domain that enhance binding to Fc receptors have been reported, for example, S239D/A330L/I332E, F243L, and G236A. Additionally or alternatively, mutations to the Fc region that increase the circulating half-life of a disclosed coronavirus-binding peptide may be incorporated into the structure. For example, mutations to engineer the pH-dependent interaction of the Fc domain with FcRn to increase affinity at pH 6.0 while retaining minimal binding at pH 7.4, can increase half-life and improve efficacy under physiological conditions. Exemplary mutations that may be incorporated in order to enhance C1q receptor or Fc receptor binding are shown in the table below.

TABLE 4
Potential Fc Mutations

| Mutation | Mechanism of Action | Effect |
|---|---|---|
| S267E | Enhance C1q binding | Increase CDC |
| H268F/S324T | Enhance C1q and Fc receptor | Increase CDC, ADCC, |
| | binding | ADCP |
| H268F/S324T/G236A/I332E | Enhance C1q and Fc receptor | Increase CDC, ADCC, |
| | binding | ADCP |
| H268F/S324T/G236D/I332E | Enhance C1q and Fc receptor | Increase CDC, ADCC, |
| | binding | ADCP |
| K326A/E333A | Enhance C1q and Fc receptor | Increase CDC, ADCC |
| | binding | |
| E345R | Enhance multimerization | Increase CDC, ADCC, |
| | | ADCP |
| M252Y/S254T/T256E | Enhanced Fc receptor | Increased half-life |
| | binding at pH 6.0 | |
| T250Q/M428L | Enhanced Fc receptor | Increased half-life |
| | binding at pH 6.0 | |
| P230S/N315D/M428L/N434Y | Enhanced Fc receptor | Increased half-life |
| | binding at pH 6.0 | |

In some embodiments, the disclosed binding proteins may be conjugated to polyethylene glycol (PEG) and/or albumin, which may increase the half-life and decrease the potential immunogenicity of the peptide.

The disclosed binding proteins may bind to a conserved coronavirus epitope as disclosed herein (e.g., one of SEQ ID NOs: 2-25) with a high affinity. For example, the disclosed binding proteins and antibodies can have a K_{D} of at least 3.0×10⁻⁸, at least 2.5×10⁻⁸, at least 2.0×10⁻⁸, at least 1.5×10⁻⁸, at least 1.0×10⁻⁸, at least 0.5×10⁻⁸, at least 9.95×10⁻⁹, at least 9.90×10⁻⁹, at least 9.85×10⁻⁹, at least 9.80×10⁻⁹, at least 9.75×10⁻⁹, at least 9.70×10⁻⁹, at least 9.65×10⁻⁹, at least 9.60×10⁻⁹, at least 9.55×10⁻⁹, at least 9.5×10⁻⁹, at least 9.45×10⁻⁹, at least 9.40×10⁻⁹, at least 9.35×10⁻⁹, at least 9.30×10⁻⁹, at least 9.25×10⁻⁹, at least 9.20×10⁻⁹, at least 9.15×10⁻⁹, at least 9.10×10⁻⁹, at least 9.05×10⁻⁹, at least 9.0×10⁻⁹, at least 8.95×10⁻⁹, at least 8.90×10⁻⁹, at least 8.85×10⁻⁹, at least 8.80×10⁻⁹, at least 8.75×10⁻⁹, at least 8.70×10⁻⁹, at least 8.65×10⁻⁹, at least 8.60×10⁻⁹, at least 8.55×10⁻⁹, at least 8.5×10⁻⁹, at least 8.45×10⁻⁹, at least 8.40×10⁻⁹, at least 8.35×10⁻⁹, at least 8.30×10⁻⁹, at least 8.25×10⁻⁹, at least 8.20×10⁻⁹, at least 8.15×10⁻⁹, at least 8.10×10⁻⁹, at least 8.05×10⁻⁹, at least 8.0×10⁻⁹, at least 7.95×10⁻⁹, at least 7.90×10⁻⁹, at least 7.85×10⁻⁹, at least 7.80×10⁻⁹, at least 7.75×10⁻⁹, at least 7.70×10⁻⁹, at least 7.65×10⁻⁹, at least 7.60×10⁻⁹, at least 7.55×10⁻⁹, at least 7.5×10⁻⁹, at least 7.45×10⁻⁹, at least 7.40×10⁻⁹, at least 7.35×10⁻⁹, at least 7.30×10⁻⁹, at least 7.25×10⁻⁹, at least 7.20×10⁻⁹, at least 7.15×10⁻⁹, at least 7.10×10⁻⁹, at least 7.05×10⁻⁹, at least 7.0×10⁻⁹, at least 6.95×10⁻⁹, at least 6.90×10⁻⁹, at least 6.85×10⁻⁹, at least 6.80×10⁻⁹, at least 6.75×10⁻⁹, at least 6.70×10⁻⁹, at least 6.65×10⁻⁹, at least 6.60×10⁻⁹, at least 6.55×10⁻⁹, at least 6.5×10⁻⁹, at least 6.45×10⁻⁹, at least 6.40×10⁻⁹, at least 6.35×10⁻⁹, at least 6.30×10⁻⁹, at least 6.25×10⁻⁹, at least 6.20×10⁻⁹, at least 6.15×10⁻⁹, at least 6.10×10⁻⁹, at least 6.05×10⁻⁹, at least 6.0×10⁻⁹, at least 5.95×10⁻⁹, at least 5.90×10⁻⁹, at least 5.85×10⁻⁹, at least 5.80×10⁻⁹, at least 5.75×10⁻⁹, at least 5.70×10⁻⁹, at least 5.65×10⁻⁹, at least 5.60×10⁻⁹, at least 5.55×10⁻⁹, at least 5.5×10⁻⁹, at least 5.45×10⁻⁹, at least 5.40×10⁻⁹, at least 5.35×10⁻⁹, at least 5.30×10⁻⁹, at least 5.25×10⁻⁹, at least 5.20×10⁻⁹, at least 5.15×10⁻⁹, at least 5.10×10⁻⁹, at least 5.05×10⁻⁹, at least 5.0×10⁻⁹, at least 4.95×10⁻⁹, at least 4.90×10⁻⁹, at least 4.85×10⁻⁹, at least 4.80×10⁻⁹, at least 4.75×10⁻⁹, at least 4.70×10⁻⁹, at least 4.65×10⁻⁹, at least 4.60×10⁻⁹, at least 4.55×10⁻⁹, at least 4.5×10⁻⁹, at least 4.45×10⁻⁹, at least 4.40×10⁻⁹, at least 4.35×10⁻⁹, at least 4.30×10⁻⁹, at least 4.25×10⁻⁹, at least 4.20×10⁻⁹, at least 4.15×10⁻⁹, at least 4.10×10⁻⁹, at least 4.05×10⁻⁹, at least 4.0×10⁻⁹, at least 3.95×10⁻⁹, at least 3.90×10⁻⁹, at least 3.85×10⁻⁹, at least 3.80×10⁻⁹, at least 3.75×10⁻⁹, at least 3.70×10⁻⁹, at least 3.65×10⁻⁹, at least 3.60×10⁻⁹, at least 3.55×10⁻⁹, at least 3.5×10⁻⁹, at least 3.45×10⁻⁹, at least 3.40×10⁻⁹, at least 3.35×10⁻⁹, at least 3.30×10⁻⁹, at least 3.25×10⁻⁹, at least 3.20×10⁻⁹, at least 3.15×10⁻⁹, at least 3.10×10⁻⁹, at least 3.05×10⁻⁹, at least 3.0×10⁻⁹, at least 2.95×10⁻⁹, at least 2.90×10⁻⁹, at least 2.85×10⁻⁹, at least 2.80×10⁻⁹, at least 2.75×10⁻⁹, at least 2.70×10⁻⁹, at least 2.65×10⁻⁹, at least 2.60×10⁻⁹, at least 2.55×10⁻⁹, at least 2.5×10⁻⁹, at least 2.45×10⁻⁹, at least 2.40×10⁻⁹, at least 2.35×10⁻⁹, at least 2.30×10⁻⁹, at least 2.25×10⁻⁹, at least 2.20×10⁻⁹, at least 2.15×10⁻⁹, at least 2.10×10⁻⁹, at least 2.05×10⁻⁹, at least 2.0×10⁻⁹, at least 1.95×10⁻⁹, at least 1.90×10⁻⁹, at least 1.85×10⁻⁹, at least 1.80×10⁻⁹, at least 1.75×10⁻⁹, at least 1.70×10⁻⁹, at least 1.65×10⁻⁹, at least 1.60×10⁻⁹, at least 1.55×10⁻⁹, at least 1.5×10⁻⁹, at least 1.45×10⁻⁹, at least 1.40×10⁻⁹, at least 1.35×10⁻⁹, at least 1.30×10⁻⁹, at least 1.25×10⁻⁹, at least 1.20×10⁻⁹, at least 1.15×10⁻⁹, at least 1.10×10⁻⁹, at least 1.05×10⁻⁹, at least 1.0×10⁻⁹, at least 0.95×10⁻⁹, at least 0.90×10⁻⁹, at least 0.85×10⁻⁹, at least 0.80×10⁻⁹, at least 0.75×10⁻⁹ at least 0.70×10⁻⁹, at least 0.65×10⁻⁹, at least 0.60×10⁻⁹, at least 0.55×10⁻⁹, at least 0.5×10⁻⁹, at least 0.45×10⁻⁹, at least 0.40×10⁻⁹, at least 0.35×10⁻⁹, at least 0.30×10⁻⁹, at least 0.25×10⁻⁹ at least 0.20×10⁻⁹, at least 0.15×10⁻⁹, at least 0.10×10⁻⁹, at least 0.05×10⁻⁹, at least 9.5×10¹⁰, at least 9.0×10⁻¹⁰, at least 8.5×10⁻¹⁰, at least 8.0×10⁻¹⁰, or any value in between.

Any of the binding proteins or antibodies disclosed herein can be used for treating and/or preventing a coronavirus infection, such as COVID-19. Optimal doses and routes of administration may vary, such as based on the route of administration and dosage form, the age and weight of the subject, and/or the subject's condition, including the type and severity of the coronavirus infection, and can be determined by the skilled practitioner. The disclosed binding proteins can be formulated in a pharmaceutical composition suitable for administration to a subject by any intended route of administration, as discussed in more detail below.

### VI. Methods of Making Binding Proteins

While the disclosed binding proteins may be prepared using any known method of protein or antibody production, they also can be prepared using the methodologies disclosed herein. In particular, human neutralizing monoclonal antibodies or binding protein can be produced according to the following processes, rather than “humanizing” mouse or rat antibodies/peptides. In general, this process allows for the development of an effective, strong, and robust library of biologics (e.g., binding proteins) that have pharmaceutical applications with significant benefits to patients or animals in the global marketplace.

Using a parent hybridoma cell line, any one or more of four distinct and effective products can be produced: (1) a fully human neutralizing monoclonal antibody—directed against any pathogen (e.g., virus or bacteria)—through use in passive immunotherapy; (2) an effective humoral active vaccine that is safe and effective; (3) an oral mini-antibody peptide-based medication with an efficacy that is equivalent to the immunologic capacity of the monoclonal antibody produced by a parent hybridoma cell; and (4) an entry-fusion inhibitor that is immunologic in character and scope. The applications for these products are broad, effective and beneficial for therapeutic use. For example, monoclonal antibodies for therapeutic use may be made to treat coronaviruses, including β-coronaviruses like SARS-CoV-2, SARS-CoV, MERS-CoV, 229E, NL63, OC43, and HKU1, among others.

In some embodiments, the disclosed method of producing a binding protein or antibody against any one of the disclosed antigens or epitopes (i.e., any one of SEQ ID NOs: 2-25, including any one of SEQ ID NOs: 2-20) may comprises the steps of: (a) identifying an asymptomatic patient after natural infection by a target infectious agent (e.g., SARS-CoV-2 or another β-coronavirus) as a donor for obtaining immune B-lymphocytes that produce high titers of plasma neutralizing antibodies directed against the target infectious agent; (b) collecting B-lymphocytes from the patient; (c) immortalizing the human B-lymphocytes to obtain immortalized cell lines; and (d) collecting antibodies produced by the immortalized cell lines. This process may optionally include the steps of (e) stabilizing and augmenting neutralizing antibody production by the immortalized cells lines; (f) screening supernatants from the immortalized cell lines for antibody production; and (g) testing the antibodies for binding against protein components of the infectious agent. The method may further comprise one or more of epitope mapping the antibodies that tested positive for binding to the infectious agent to screen for antibodies/binding proteins that specifically bind to any one of SEQ ID NOs: 2-25; purifying the antibodies by affinity chromatographic techniques; and in vitro testing of the antibodies to confirm neutralization reactivity against the target infectious agent at physiologic concentrations.

### VII. Pharmaceutical Compositions

Also provided herein are pharmaceutical compositions comprising a disclosed antigen (SEQ ID NOs: 2-25) (e.g., for a vaccine composition) or a disclosed binding protein (specifically binding to one of SEQ ID NOs: 2-25) (e.g., for a passive immunization or therapeutic composition) and a pharmaceutically acceptable carrier or diluent. A vaccine composition as disclosed herein may include one or a plurality of peptide antigens, each comprising or consisting of a different one of SEQ ID NOs: 2-25, including one, two, three, four, five, six, seven, eight, nine, ten, 11, 12, 13, 14, 15, 16, 17, 18, 19, 20, 21, 22, 23, or all 24 of SEQ ID NOs: 2-25; one, two, three, four, five, six, seven, eight, nine, ten, 11, 12, 13, 14, 15, 16, 17, 18, or all 19 of SEQ ID NOs: 2-20; or one or more or all of SEQ ID NOs: 3, 5, 8, 10-12, 14, and 17-22, including one or more or all of SEQ ID Nos:18-20. A passive immunization composition as disclosed herein may include one or a plurality of binding proteins, each binding to a different one of SEQ ID NOs: 2-25, including binding proteins binding to one, two, three, four, five, six, seven, eight, nine, ten, 11, 12, 13, 14, 15, 16, 17, 18, 19, 20, 21, 22, 23, or all 24 of SEQ ID NOs: 2-25; binding proteins binding to one, two, three, four, five, six, seven, eight, nine, ten, 11, 12, 13, 14, 15, 16, 17, 18, or all 19 of SEQ ID NOs: 2-20; or binding proteins binding to one or more or all of SEQ ID NOs: 3, 5, 8, 10-12, 14, or 17-22, including one or more or all of SEQ ID NOs:18-20. Additionally or alternatively, a passive immunization composition may comprise a plurality of binding proteins (i.e., antibodies) that each bind to the same one of SEQ ID NOs: 2-25. As noted above, in any embodiments, the binding proteins may be monoclonal antibodies.

The disclosed pharmaceutical compositions may be formulated for any suitable route of administration, including intravenous, subcutaneous, intraperitoneal, intramuscular, or oral administration. In typical embodiments, the binding proteins are formulated for intravenous, subcutaneous, intraperitoneal, or intramuscular administration, such as in a solution, suspension, emulsion, liposome formulation, etc. More specifically, the disclosed binding proteins can be formulated for intravenous, subcutaneous, or intramuscular administration. The pharmaceutical composition can be formulated to be an immediate-release composition, sustained-release composition, delayed-release composition, etc., using techniques and excipients that are known in the art.

Pharmaceutically acceptable carriers for various dosage forms and routes of administration are known in the art. For example, solvents, solubilizing agents, suspending agents, isotonicity agents, buffers, and soothing agents for liquid preparations are known. In some embodiments, the pharmaceutical compositions include one or more additional components, such as one or more preservatives, antioxidants, colorants, sweetening/flavoring agents, adsorbing agents, wetting agents and the like.

Pharmaceutical compositions of the disclosed antigens or binding proteins can be prepared as formulations according to standard methods (see, for example, Remington's Pharmaceutical Science, Mark Publishing Company, Easton, USA). The pharmaceutical compositions generally comprise a carrier and/or additive in addition to the antigen or binding protein (e.g., antibody). For example, the pharmaceutical composition may comprise one or more surfactants (for example, PEG and Tween), excipients, antioxidants (for example, ascorbic acid), preservatives, stabilizers, buffering agents (for example, phosphoric acid, citric acid, and other organic acids), chelating agents (for example, EDTA), suspending agents, isotonizing agents, binders, disintegrators, lubricants, fluidity promoters, corrigents, light anhydrous silicic acid, lactose, crystalline cellulose, mannitol, starch, carmelose calcium, carmelose sodium, hydroxypropylcellulose, hydroxypropylmethylcellulose, polyvinylacetaldiethylaminoacetate, polyvinylpyrrolidone, gelatin, medium chain fatty acid triglyceride, polyoxyethylene hydrogenated castor oil 60, sucrose, carboxymethylcellulose, corn starch, and inorganic salt. The pharmaceutical composition may comprise one or more other low-molecular-weight polypeptides or proteins, such as serum albumin, gelatin, immunoglobulin, or amino acids such as glycine, glutamine, asparagine, arginine, and lysine.

When the antigens or binding proteins are prepared as an aqueous solution for injection, the antigen or binding protein may be formulated in an isotonic solution containing, for example, physiological saline, dextrose, or other excipients or tonifiers. The tonifier may include, for example, D-sorbitol, D-mannose, D-mannitol, and sodium chloride. In addition, appropriate solubilizing agents, for example, alcohols (for example, ethanol), polyalcohols (for example, propylene glycols and PEGs), and non-ionic detergents (polysorbate 80 and HCO-50) may be used concomitantly.

The disclosed antigens and binding proteins may be formulated for administration by injection or infusion, such as an intravenous injection or infusion, an intramuscular injection, or a subcutaneous injection. Alternatively, the disclosed antigens or binding proteins may be formulated for oral administration.

Any of the pharmaceutical compositions disclosed herein can be used for inducing an immune response to, or treating and/or preventing a coronavirus infection, such as COVID-19. Optimal doses and routes of administration may vary, such as based on the route of administration and dosage form, the age and weight of the subject, and/or the subject's condition, including the type and severity of the coronavirus infection, and can be determined by the skilled practitioner.

### VIII. Treatment and Prevention of Coronavirus Infection

COVID-19 and SARS-CoV-2 can cause serious infection and death. The discovery of numerous variants, such as B.1.1.7 (first identified in the United Kingdom), B.1.351 (first identified in South Africa), and P.1(identified in Brazil) concern scientists because of emerging data suggesting their increased transmissibility. Variants, particularly those with changes in the spike protein sequence, are alarming because changes in this protein may reduce vaccine effectiveness. Indeed, the B.1.351 variant may be partially or fully resistant to certain SARS-CoV-2 monoclonal antibodies currently authorized for use as therapeutics in the United States. The disclosed antigens and binding protein address this concern by providing highly conserved antigens and epitopes that are likely to remain conserved even across variant strains of SARS-CoV-2.

The present disclosure provides methods of inducing an immune response against coronaviruses, such as SARS-CoV-2, by administering an antigen as disclosed herein, i.e., an antigen that comprises or consists of a peptide having one of the sequences in Tables 1 or 2 (i.e., selected from SEQ ID NOs: 2-25, including selected from SEQ ID NOs: 2-20, selected from SEQ ID NOs: 3, 5, 8, 10-12, 14, and 17-22, and selected from SEQ ID NOs:18-20), in the form of a vaccine as discussed above. The present disclosure also provides uses of the disclosed antigens and pharmaceutical compositions (vaccines) for inducing an immune response to coronaviruses, such as SARS-CoV-2. The immune response may be effective to reduce the risk of infection by coronaviruses, such as SARS-CoV-2. The immune response may be effective to partially or fully protect against infection, such as by preventing infection or reducing the viral load if the subject does get infected.

The present disclosure also provides methods of treatment and prevention of coronavirus infections, such as SARS-CoV-2 infections (e.g., COVID-19) by administering a binding protein that specifically binds to at least one of the epitopes disclosed herein, i.e., having one of the sequences in Tables 1 or 2 (i.e., selected from SEQ ID NOs: 2-25, including selected from SEQ ID NOs: 2-20, selected from SEQ ID NOs: 3, 5, 8, 10-12, 14, and 17-22, and selected from SEQ ID NOs:18-20), (SEQ ID NOs: 2-20). The present disclosure also provides uses of the disclosed binding proteins and pharmaceutical compositions for treating or preventing coronavirus infections, such as SARS-CoV-2 infections (e.g., COVID-19). As noted above, in any embodiments, the binding proteins may be monoclonal antibodies.

The disclosed methods comprise administering to a subject an effective amount of one or more of the antigens or binding proteins or pharmaceutical compositions disclosed herein. Administration may be performed via intravenous, intra-arterial, intramuscular, subcutaneous, or intradermal injection. In some embodiments, the subject may be at risk of exposure to a coronavirus, such as SARS-CoV-2. In some embodiments, the administration of the antigen prevents the subject from developing a coronavirus infection (e.g., COVID-19). In some embodiments, the administration of the antigen reduces the risk the subject will develop a severe coronavirus infection (e.g., COVID-19), such as reducing the risk of infection requiring hospitalization. In some embodiments, the subject may have previously been exposed to a coronavirus, such as SARS-CoV-2. In some embodiments, particularly embodiments using binding proteins, the subject may have an active infection (e.g., COVID-19) which may be treated as a result of the administration. In some embodiments, the administration of the binding protein prevents the subject from developing a coronavirus infection (e.g., COVID-19). In some embodiments, the effective amount of a binding protein is sufficient to reduce circulating viral load and/or to reduce, ameliorate, or eliminate one or more symptoms or effects of a coronavirus infection, such as COVID-19. In some embodiments, the effective amount of a binding protein is effective to prevent binding of a SARS-CoV-2 S protein to an ACE2 receptor. The specific amount of antigen or binding protein administered may depend on one or more of the age and/or weight of the subject and/or the stage or severity of the disease and/or the dosage form and route of administration, and can be determined by the skilled practitioner.

For the purposes of the disclosed methods and uses, treatment and/or prevention of all strains and variants of SAR-CoV-2 are specifically contemplated.

Dosage regimens can be adjusted to provide the optimum desired response. For example, in some embodiments, a single bolus of an antigen or binding protein may be administered, while in some embodiments, several doses may be administered over time, or the dose may be proportionally reduced or increased as indicated by the situation. In some embodiments, a subject may be administered more than one distinct antigen or binding protein, such as two or three or more distinct antigens or antibodies that each bind to different epitopes disclosed herein.

### IX. Detection of Coronavirus Infection

As noted above the peptide antigens and binding proteins thereto described herein also are useful for detecting coronavirus infection, such as SARS-CoV-2 infection.

The currently available tests for SARS-CoV-2 infection can be classified into two categories: diagnostic tests and surveillance tests. Diagnostic tests detect a component of the virus in a sample, typically taken from the nasal cavity, throat, or saliva. The test format can be a molecular test that detects viral RNA or an antigen test that detects viral protein. Molecular tests also are called Nucleic Acid Amplification Tests (NAATs), and involve amplifying nucleic acids present in the sample until they are detectable. The polymerase chain reaction (PCR) is viewed as the “gold standard” for diagnostic tests, but can show false-negative results at early stages of infection. RT-PCR is expensive, requires expert handling, and takes about four hours to complete the assay. Currently available antigen tests are less expensive, and offer nearly instant results without requiring skilled personnel, but also are less sensitive, so the rate of false negatives is high, and false positives also have been observed.

The fast evolution of SARS-CoV-2 variants having mutations in their genomic and protein sequences poses another problem in the development of effective methods for detecting infection. Mutations can impact test performance if the mutation impairs or prevents the test reagent from being able to detect the virus. The impact of mutations on a test's performance may be influenced by several factors, including the sequence of the variant (including the number, identity and location of mutations), the design of the test, and the prevalence of the variant in the population. For example, tests with single targets are more likely to fail to detect new variants. On the other hand, tests with multiple targets (e.g., a PCR test designed to detect more than one section of the SARS-CoV-2 genome or an antigen test designed to detect more than one region of the spike protein) are more likely to be able to detect new variants. For example, one marketed molecular test able to detect other variants is expected to fail to detect the SARS-CoV-2 Omicron variant (B.1.1.529) due to a nine-nucleotide deletion in the N-gene, spanning positions 28370-28362, while a marketed two-target antigen test able to detect other variants is expected to fail to detect the Omicron variant due to deletions at amino acid positions 69-70 and mutations at nucleotide positions 23599 (T to G) and 23604 (C to A).

The peptide antigens and binding proteins described herein offer significant advantages in this context due to the highly conserved nature of the corresponding epitopes. As discussed above, the epitopes of SEQ ID NOs: 2-20 have been confirmed to be conserved in the original Wuhan strain and all variants screened, including Alpha, Beta, Gamma, Delta, Lamda, Mu, and Omicron, and the epitopes of SEQ ID NOs: 21-25 have been confirmed to be conserved in the Delta and Omicron variants. This indicates that detection tests targeting these epitopes will be able to detect infection by all currently known variants, as well as future variants. Test can involve detecting SEQ ID NOs: 2-25 using a binding protein or, alternatively, can involve detecting antibodies that bind to SEQ ID NOs: 2-25, as described herein.

Thus, provided herein are an “antigen test” and kit for detecting coronavirus infection, such as SARS-CoV-2 infection, comprising detecting any one or more or all of SEQ ID NOs: 2-25 in a sample obtained from a subject, including detecting any one, two, three, four, five, six, seven, eight, nine, ten, 11, 12, 13, 14, 15, 16, 17, 18, 19, 20, 21, 22, 23, or all 24 of SEQ ID NOs: 2-25 in a sample. For the purposes of such antigen tests, an ELISA (i.e., enzyme-linked immunoassay) is a particularly useful format, but other known methods in the art may be used, such as Western blotting, dot blotting, immunohistochemistry, immunofluorescence, immunoprecipitation, immunoelectrophoresis, or mass-spectrometry, and any other assay format that can detect the presence of any one or more or all of SEQ ID NOs: 2-25 in a sample.

For example, an ELISA-based method of detecting SARS-CoV-2 infection may comprise contacting a sample obtained from a subject with one or more probe binding proteins or antibodies that each specifically bind any one or more or all of SEQ ID NOs: 2-25, and detecting binding between the probe binding proteins/antibodies and any SARS-CoV-2 antigen present in the sample. In some embodiments, such a method may comprise using a single monoclonal antibody that is specific for any one of SEQ ID NOs: 2-25 or a panel of binding proteins/antibodies that each specifically binds one of SEQ ID NOs: 2-25, typically where each specifically binds a different one of SEQ ID NOs: 2-25, such as a panel of 2-24 binding proteins/antibodies where each specifically binds a different one of SEQ ID NOs: 2-25. In such embodiments, the probe antibodies or binding proteins that bind to the SARS-CoV-2 antigens may be bound to a solid substrate (e.g., a plate, well, slide, bead, strip, etc.) and a biological sample obtained from a subject (such as an individual suspected of having or having been exposed to SARS-CoV-2) may be applied to the substrate. If a target antigen (i.e., antigens comprising or consisting of SEQ ID NOs: 2-25) is present in the sample and a probe antibody/binding protein that is specific for the target antigen is bound to the substrate, then the target antigen will be bound. The biological sample can then be removed and the substrate may be washed to remove any unbound protein or debris. Next, a detection antibody that also binds to the target antigen may be contacted to the substrate. The detection antibody typically is detectably labeled, such that it can be detected as evidence of the presence of the target antigen in the sample. Detectable labels that can be used for this purpose are known in the art and can include, but are not limited to, a fluorophore (e.g., FTIC, rhodamine, GFP, lanthanide, etc.), a chromogen, a chemiluminescent agent, an enzymatic label (e.g., luciferase, horseradish peroxidase, alkaline phosphatase), an acridinium moiety, a radiolabel, a colorometric label, a magnetic agent, or a metal (e.g., a gold particle).

Also provided herein is an antibody test for detecting coronavirus infection, such as SARS-CoV-2 infection, comprising detecting antibodies to any one or more or all of SEQ ID NOs: 2-25 in a sample obtained from a subject, including antibodies to any one, two, three, four, five, six, seven, eight, nine, ten, 11, 12, 13, 14, 15, 16, 17, 18, 19, 20, 21, 22, 23, or all 24 of SEQ ID NOs: 2-25 in a sample. Antibodies to each of SEQ ID NOs: 2-25 can be detected using a peptide antigen as described herein as a probe. Thus, a method of detecting SARS-CoV-2 infection may comprise contacting a sample obtained from a subject with one or more peptide antigens, each comprising or consisting of one of SEQ ID NOs: 2-25. In some embodiments, such a method comprises using a panel of peptide antigens, each comprising or consisting of one of SEQ ID NOs: 2-25, typically where each comprises or consists of a different one of SEQ ID NOs: 2-25, such as a panel of 2-24 peptide antigens, typically where each comprises or consists of a different one of SEQ ID NOs: 2-25. For the purposes of such embodiments, the probe antigen(s) may be bound to a substrate (e.g., a plate, well, slide, bead, strip, etc.) either directly or indirectly (e.g., via a polymer, such as polyethylene glycol (PEG), a peptide linker, or a protein, such as an antibody). The substrate may be contacted with a biological sample obtained from a subject (such as an individual suspected of having or having been exposed to SARS-CoV-2). If there are antibodies present in the sample that bind to any one of the probe antigens, then the presence of such antibodies can be detected, such as by removing the sample and washing the substrate to remove any unbound protein or debris, and then contacting the substrate with a detection antibody that binds to antibodies from the sample (e.g., human IgE or IgD). The detection antibody typically is detectably labeled, as discussed above.

The present disclosure also provides methods for detection of coronavirus, and specifically, SARS-CoV-2, by detecting the presence of nucleic acid sequence(s) that encode any one or more or all of SEQ ID NOs: 2-25. Such methods include, but are not limited to, RT-qPCR, RT-PCR, RNA-seq, Northern blotting, Serial Analysis of Gene Expression (SAGE), or DNA or RNA microarrays. The starting material for detection of polynucleotides encoding the disclosed biomarkers may be genomic DNA, cDNA, RNA or mRNA. In such embodiments, nucleic acid primers and, optionally, nucleic acid probes, may be designed to specifically amplify and detect the nucleic acid sequences that encode any one or more or all of SEQ ID NOs: 2-25. Primers and probes may comprise a detectable label or a plurality of detectable labels. The detectable label associated with the primer or probe can generate a detectable signal directly. Additionally, the detectable label associated with the primer or probe can be detected indirectly using a reagent, wherein the reagent includes a detectable label, and binds to the label associated with the probe.

Detectably labeled nucleic acid primers and probes can be used to monitor the amplification of a target nucleic acid sequence (e.g., nucleic acid sequences that encode any one or more or all of SEQ ID NOs: 2-25). In some embodiments, detectably labeled primers or probes present in an amplification reaction are suitable for monitoring the amount of amplicon(s) produced as a function of time. Examples of such probes include, but are not limited to, the 5′-exonuclease assay (TAQMAN® probes described herein (see also U.S. Pat. No. 5,538,848) various stem-loop molecular beacons (see for example, U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, for example, Kubista et al., 2001, SPIE 4264:53-58), non-FRET probes (see, for example, U.S. Pat. No. 6,150,097), Sunrise®/Amplifluor™ probes (U.S. Pat. No. 6,548,250), stem-loop and duplex Scorpion probes (Solinas et al., 2001, Nucleic Acids Research 29:E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem. Res. Toxicol. 15:118-126; and Yu et al., 2001, J. Am. Chem. Soc 14:11155-11161. In some embodiments, the detectable label is a fluorophore. Suitable fluorescent moieties include but are not limited to the following fluorophores working individually or in combination: 4-acetamido-4′-isothiocyanatostilbene-2,2′disulfonic acid; acridine and derivatives: acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120),7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)amino fluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescem (TET); fiuorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron®Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; tetramethyl rhodamine isothiocyanate (TRITC); and VIC®. Detector probes can also comprise sulfonate derivatives of fluorescenin dyes with S03 instead of the carboxylate group, phosphoramidite forms of fluorescein, phosphoramidite forms of CY 5 (commercially available for example from Amersham).

Nucleic acid primers or probes may be designed to selectively hybridize to any portion of a nucleic acid sequence encoding any one or more or all of SEQ ID NOs: 2-25. Methods for preparing nucleic acid primers or probes are well known in the art.

Accordingly, the present disclosure provides in vitro methods of analyzing a biological sample obtained from a subject, comprising contacting the sample with a binding protein that specifically binds to a SARS-CoV-2 peptide antigen selected from SEQ ID NOs: 2-25, and detecting binding between the binding protein and any SARS-CoV-2 antigen present in the sample. The methods may comprise contacting the sample with a panel of from 2 to 24 binding proteins that each specifically binds to a different SARS-CoV-2 peptide antigen selected from SEQ ID NOs: 2-25, and detecting binding between the binding proteins and any SARS-CoV-2 antigen present in the sample.

The present disclosure also provides in vitro methods of analyzing a biological sample obtained from a subject, comprising contacting the sample with a SARS-CoV-2 peptide antigen comprising or consisting of an amino acid sequence selected from SEQ ID NOs: 2-25, and detecting binding between the peptide antigen and any anti-SARS-CoV-2 antibodies present in the sample. The methods may comprise contacting the sample with a panel of from 2 to 24 peptide antigens each comprising or consisting of a different an amino acid sequence selected from SEQ ID NOs: 2-25, and detecting binding between the peptide antigen and any anti-SARS-CoV-2 antibodies present in the sample.

The present disclosure also provides in vitro methods of analyzing a biological sample obtained from a subject, comprising extracting nucleic acids from the biological sample, contacting the extracted nucleic acids with a pair of primers that specifically amplify a nucleic acid sequence encoding any one of SEQ ID NOs: 2-25, and detecting the presence of the amplified nucleic acid sequence if present in the sample. The methods may comprise contacting the sample with a panel of from 2 to 24 primer pairs each specific for a different nucleic acid sequence that encodes an amino acid sequence selected from SEQ ID NOs: 2-25, and detecting amplification of each nucleic acid sequence if present in the sample.

As discussed above, the epitopes of SEQ ID NOs: 2-25 to be targeted for detection can be selected depending on the aim of the analysis. For examp(Sle, epitopes of SEQ ID NOs: 2-25 shown to be highly conserved across all variants (such as SEQ ID NO:13) can be used to detect infection by any variant, while epitopes shown to present in fewer than all variants or in only one variant can to be targeted to detect infection by that variant or those variants (such as SEQ ID NO:14 for the original Wuhan strain).

In some embodiments of the disclosed methods, the sample may be selected from saliva, nasal fluid, nasal cells, throat cells, blood, plasma, serum, urine, and feces. However, the sample is not necessarily limited to these sample types. In some instances, a blood sample, a plasma sample, a serum sample, or a tissue sample may be appropriate. In some instances, the sample may comprise urine or feces, which may be useful for epidemiological studies and public health tracking that relies on wastewater. Thus, in some embodiments the sample comprises biological samples obtained from a plurality of subjects.

In some embodiments of the disclosed methods, the subject is suspected of having a SARS-CoV-2 infection, has been exposed to SARS-CoV-2, or is suspected of having been exposed to SARS-CoV-2. In some embodiments of the disclosed methods, the method may further comprise determining that the subject is infected with SARS-CoV-2 when binding or amplification is detected.

As noted above, in some embodiments, the sample comprises biological samples obtained from a plurality of subjects. Some such embodiments further comprise determining a level of infection in the plurality of subjects.

The present disclosure additionally provides kits for implementing any of the foregoing methods of detection.

For example, the present disclosure provides kits comprising at least one binding protein that specifically binds to a peptide comprising or consisting of any one of SEQ ID NOs: 2-25, a solid substrate to which the at least one binding protein is attached, and a second detectably labeled antibody that specifically binds to the peptide to which the at least one binding protein specifically binds. Similarly, the present disclosure provides kits comprising at least one peptide comprising or consisting of any one of SEQ ID NOs: 2-25, a solid substrate to which the at least one peptide is attached, and a detectably labeled antibody that specifically binds to IgE or IgD, wherein the IgE or IgD are optionally human. The solid substrate can be selected from a bead, a plate, a well, a dish, a slide, or a strip.

The present disclosure also provides kits comprising at least one primer pair capable of specifically amplifying a nucleic acid sequence that encodes a peptide selected from any one of SEQ ID NOs: 2-25, wherein: (a) at least one primer of the primer pair is detectably labeled; or (b) the kit further comprises a detectably labeled probe that hybridizes to the nucleic acid sequence amplified by the primer pair. In some embodiments, the kit may comprise 1, 2, 3, 4, 5, 6, 7, 8, 9, 10, 11, 12, 13, 14, 15, 16, 17, 18, 19, 20, 21, 22, 23, or 24 primer pairs, each primer pair being capable of specifically amplifying a different peptide selected from SEQ ID NOs: 2-25, wherein: (a) at least one primer of each primer pair is detectably labeled; or (b) the kit further comprises a detectably labeled probe that hybridizes to each nucleic acid sequence amplified by primer pairs included therein.

The following examples are given to illustrate the present disclosure. It should be understood that the invention is not to be limited to the specific conditions or details described in these examples.

### EXAMPLES

### Example 1—Identification of Highly Conserved Antigens and Epitopes

This example describes the analytical methods used to identify the disclosed putative epitopes and antigens of SEQ ID NOs:2-20. The identification process used computational prediction and machine learning (ML) approaches to identify optimal targets and assess antigenicity.

### Data Collection

A protein antigen dataset was created to train an artificial intelligence model to predict and map other linear, sequential, conserved and neutralizable B-cell epitopes within the SARS-CoV-2 S protein, specifically the S1 and S2 subunits. Coronavirus S protein sequences were collected and multiple sequence alignments were performed to identify conserved sequences. Additional epitope databases (e g , IEDB and AntiJen, BciPep, Epitome, SDAP, FLAVIdB, and Influenza Sequence and Epitope Database) were used to obtain independent and relevant data points to ensure unbiased training.

### Curating Dataset

All identical epitopes (sequence and structure based) and non-immunogenic peptides were removed, so as to achieve unique experimentally proved epitopes. Generally, majority of B-cells epitope have length less than or equal to 20 amino acid, hence in this analysis all the epitopes having length more than 20 residues were removed. For training of machine learning technique, it was necessary to have fixed length patters whereas B-cell epitopes have varying length, hence if the epitope length were less than 20 amino acids, then the length was increased by introducing equal number of residues at both terminals derived from its original antigenic sequence. To generate a negative dataset, non-epitopes were created using random peptides of length 20 residues from the proteins in Swiss-Prot. All the random peptides that are identical to B-cells epitopes were excluded. Next, the unique epitope data was scored based on biophysical and biochemical metrics such as computed volume, polarity, hydrophobicity, linearity, activity and immunogenicity. These metrics served as dimensions across which regression, classification methods were performed. After a cleaned and labelled dataset was generated a portion of the dataset was used for training purposes. In order to achieve five-fold cross-validation approach. Multilayer perceptron method was implemented. The dataset was randomly divided into five subsets each containing an equal number of epitopes data. The three of the subsets were used for training purpose and from remaining two subsets each subset was used for validation and testing. This process was repeated five times so that each set was used once for testing. The final prediction results gave the average of five testing sets.

### ANN Model Training

To predict the probability that a given antigen residue is part of an epitope, artificial neural network (Jordon Network) was implemented using Keras and TensorFlow. The networks were trained using back-propagation algorithm and with various window lengths from 10 to 20 residues. The target output consists of a single binary number with one or zero (B-cell epitopes or non-epitopes). At the beginning of each simulation, the weights were initialized with random values and the training was carried out by using error back-propagation, with a sum of square error function. In each cycle of the training, the magnitude of the error sum in the test and training set were monitored and the ultimate number of cycles were determined when the network converges. Also a cut off value for each network was set up, which was used to compare the output produced by the network. Thus, when the output value was greater than the threshold value, then that peptide was predicted as B-cell epitope, otherwise as a non-epitope. Additionally, each amino acid composition, along with other parameters such as computed volume, polarity, hydrophobicity, linearity, neutralizing activity and immunogenicity were used for prediction purpose. We accurately predict the likelihood of each residue being an epitope candidate. To ensure the capability of the model in determining the correct epitope for a given antibody, these models with two hidden layers were implemented. Parameters of prediction include amino acid composition, exposed donors/receptors, hydrophobicity, aromatic/positive/negative residues, size, antigen patch density and structural conjoint triads to represent the specified protein sequences by considering not only the composition of amino acids but also the neighbor relationships in that sequence. Once the system was trained on the curated database, the blind dataset evaluation was performed using 1) clinically validated immunogenic proteins, 2) IgE epitopes of allergenic proteins (SDAP), and none of these datasets were used in the training or testing.

### Statistical Evaluation of Linear Epitope Dataset

FASTA sequences of the coronavirus virus peptides were inputted into the system and epitope predictions with percentage of conservedness, neutralizing activity and immunogenicity were established. Each model was tuned by comparing prediction accuracy from the test set to predict the accuracy on the validation set. Models that perform the best were scored on the test set and evaluated using metrics such as precision, recall, true positive rate, false positive rate, and ROC-area under the curve using sci-kitlearn, Keras, and TensorFlow. Once potential candidates were identified, the performance for each antigen in terms of the area under the receiver operation curve, the positive predictive rate and the true positive rate of the top predictions was evaluated for immune recognition.

Thus, SEQ ID NOs: 2-20 were identified as highly conserved epitopes having the conservation across isolates reported in Tables 1 and 2.

### Example 2—Confirmation of Disclosed Antigens and Epitopes

Utilizing artificial intelligence, over 2 million isolates of SARS-CoV-2 were analyzed, and the 19 conserved immutable sites on SARS-CoV-2 (of SEQ ID NOs: 2-20) were confirmed. The conserved epitopes of SEQ ID NOs: 2-20 were confirmed to be conserved in the original Wuhan strain and the Alpha, Beta, Gamma, Delta, Lambda, Mu, and Omicron variants. The conserved sites remained conserved regardless of the mutations, thus making these region ideal for vaccine development, antibody targeting, and diagnostic development.

This extensive artificial intelligence analysis has been done in three phases. Phase I analyzed 57,500 SARS-CoV-2 isolates and identified the 19 conserved sites of SEQ ID NOs: 2-20. The conserved sites were confirmed to be conserved in the original Wuhan strain and the Alpha, Beta, and Gamma variants. In Phase-2, the 19 identified conserved sites of SEQ ID NOs: 2-20 were screened against further isolates to confirm their presence in Delta, Lambdas and Mu variants. Phase 3 analyzed 2 million SARS-CoV-2 isolates and confirmed the 19 conserved sites of SEQ ID NOs: 2-20. The conserved sites were confirmed to be conserved in the original Wuhan strain and the Alpha, Beta ,Gamma, Delta, Lambda, Mu, and Omicron variants.

### Example 3—Identification of Additional Antigens and Epitopes

Utilizing artificial intelligence, 2.8 million isolates of SARS-CoV-2 Delta and Omicron variants were analyzed as described above, and SEQ ID NOs: 21-25 were identified as conserved epitopes across the isolates, as reported in Table 1 above.

What is claimed is:

1. An isolated peptide antigen comprising or consisting of an amino acid sequence of any one of SEQ ID NOs: 2-20.

2. An isolated peptide antigen comprising or consisting of an amino acid sequence of any one of SEQ ID NOs: 21-25, optionally wherein the amino acid sequence is selected from any of SEQ ID NOs: 21 and 22.

3. The isolated peptide antigen of claim 1, wherein the amino acid sequence is selected from any of SEQ ID NOs: 3, 5, 8, 10-12, 14, or 17-20, optionally wherein the amino acid sequence is selected from any of SEQ ID NOs: 18-20.

4. The isolated peptide antigen of any one of claims 1-3, wherein the peptide is recombinant.

5. A vaccine composition comprising a peptide antigen of any one of claims 1-4 and a pharmaceutically acceptable carrier.

6. An isolated binding protein that binds to a conserved epitope of a coronavirus S protein, wherein the conserved epitope comprises the amino acid sequence of any one of SEQ ID NOs: 2-20.

7. The isolated binding protein of claim 6, wherein the conserved epitope is selected from any one of SEQ ID NOs: 2-12.

8. The isolated binding protein of claim 6, wherein the conserved epitope is selected from any one of SEQ ID NOs: 13-20.

9. The isolated binding protein of claim 6, wherein the conserved epitope is selected from any one of SEQ ID NOs: 3, 5, 8, 10-12, 14, or 17-20, optionally wherein the conserved epitope is selected from any of SEQ ID NOs:18-20.

10. An isolated binding protein that binds to a conserved epitope of a coronavirus S protein, wherein the conserved epitope comprises the amino acid sequence of any one of SEQ ID NOs: 21-25, optionally wherein the conserved epitope is selected from any of SEQ ID NOs: 21 and 22.

11. The isolated binding protein of any one of claims 6-10, wherein the binding protein prevents the coronavirus S protein from binding to an angiotensin-converting enzyme 2 (ACE2) receptor.

12. The isolated binding protein of any one of claims 6-11, wherein the coronavirus is SARS-CoV-2 or a variant of SARS-CoV-2.

13. The isolated binding protein of any one of claims 6-12, wherein the binding protein is an antibody or an antibody fragment, optionally wherein the binding protein is a monoclonal antibody.

14. A method of reducing the risk of a coronavirus infection in a subject, comprising administering to the subject an effective amount of an isolated peptide antigen according to any one of claims 1-4 or a vaccine according to claim 5.

15. A method of treating, preventing, or reducing the risk of a coronavirus infection in a subject, comprising administering to the subject a therapeutically effective amount of an isolated binding protein according to any one of claims 6-13.

16. The method of claim 14 or 15, wherein the isolated peptide antigen or isolated binding protein is administered by subcutaneous or intramuscular injection.

17. The method of any one of claims 14-16, wherein the coronavirus is SARS-CoV-2 or a variant of SARS-CoV-2.

18. An isolated peptide antigen according to any one of claims 1-4 for inducing an immune response to a coronavirus.

19. An isolated binding protein according to any one of claims 6-13 for treating, preventing, or reducing the risk of a coronavirus infection in a subject in need thereof.

20. The isolated peptide antigen or binding protein of claim 18 or 19, wherein the coronavirus is SARS-CoV-2 or a variant of SARS-CoV-2.

21. Use of an isolated peptide antigen according to any one of claims 1-4 in the preparation of a vaccine for inducing an immune response to a coronavirus.

22. Use of an isolated binding protein according to any one of claims 6-13 in the preparation of a medicament for treating, preventing, or reducing the risk of a coronavirus infection in a subject in need thereof.

23. The use of claim 21 or 22, wherein the coronavirus is SARS-CoV-2 or a variant of SARS-CoV-2.

24. A method of preparing an antibody that binds to a peptide antigen of any one of claims 1-4, comprising:
(a) identifying an asymptomatic patient that has been infected with a coronavirus as a donor for obtaining immune B-lymphocytes that produce high titers of coronavirus-neutralizing antibodies;
(b) collecting the B-lymphocytes from the patient;
(c) immortalizing the B-lymphocytes;
(d) collecting antibodies produced by the immortalized B-lymphocytes; and
(e) screening the antibodies for binding to the peptide antigen of any one of claims 1-4.

25. The method of claim 24, further comprising testing the antibodies for binding to SARS-CoV-2.

26. The method of claim 25, further comprising epitope mapping the antibodies that tested positive for binding to SARS-CoV-2.

27. The method of any one of claims 24-26, wherein immortalizing the B-lymphocytes comprises fusing a B-lymphocyte with a heteromyeloma cell in order to produce a heterohybridoma cell.

28. An in vitro method of analyzing a biological sample obtained from a subject, comprising contacting the sample with a binding protein that specifically binds to a SARS-CoV-2 peptide antigen selected from SEQ ID NOs: 2-25, and detecting binding between the binding protein and any SARS-CoV-2 antigen present in the sample; optionally, wherein the SARS-CoV-2 peptide antigen comprises or consists of any one of SEQ ID NOs: 2-20.

29. The method of claim 28, comprising contacting the sample with a panel of from 2 to 24 binding proteins that each specifically binds to a different SARS-CoV-2 peptide antigen selected from SEQ ID NOs: 2-25, and detecting binding between the binding proteins and any SARS-CoV-2 antigen present in the sample.

30. An in vitro method of analyzing a biological sample obtained from a subject, comprising contacting the sample with a SARS-CoV-2 peptide antigen comprising or consisting of an amino acid sequence selected from SEQ ID NOs: 2-25, and detecting binding between the peptide antigen and any anti-SARS-CoV-2 antibodies present in the sample; optionally, wherein the SARS-CoV-2 peptide antigen comprises or consists of any one of SEQ ID NOs: 2-20.

31. The method of claim 30, comprising contacting the sample with a panel of from 2 to 24 peptide antigens each comprising or consisting of a different an amino acid sequence selected from SEQ ID NOs: 2-25, and detecting binding between the peptide antigen and any anti-SARS-CoV-2 antibodies present in the sample.

32. An in vitro method of analyzing a biological sample obtained from a subject, comprising extracting nucleic acids from the biological sample, contacting the extracted nucleic acids with a pair of primers that specifically amplify a nucleic acid sequence encoding a peptide of any one of SEQ ID NOs: 2-25, and detecting the presence of the amplified nucleic acid sequence if present in the sample; optionally, wherein the peptide comprises or consists of any one of SEQ ID NOs: 2-20.

33. The method of claim 32, comprising contacting the sample with a panel of from 2 to 24 primer pairs that each specifically amplify a nucleic acid sequence that encodes a different amino acid sequence selected from SEQ ID NOs: 2-25, and detecting amplification of each nucleic acid sequence if present in the sample.

34. The method of any one of claims 28-33, wherein the sample is selected from saliva, nasal fluid, nasal cells, throat cells, blood, plasma, serum, urine, and feces.

35. The method of any one of claims 28-34, where the subject is suspected of having a SARS-CoV-2 infection, has been exposed to SARS-CoV-2, or is suspected of having been exposed to SARS-CoV-2.

36. The method of any one of claims 28-35, further comprising determining that the subject is infected with SARS-CoV-2 when binding or amplification is detected.

37. A kit comprising at least one binding protein that specifically binds to a peptide comprising or consisting of any one of SEQ ID NOs: 2-25, a solid substrate to which the at least one binding protein is attached, and a detectably labeled antibody that specifically binds to the peptide to which the at least one binding protein specifically binds; optionally, wherein the peptide comprises or consists of any one of SEQ ID NOs: 2-20.

38. A kit comprising at least one peptide comprising or consisting of any one of SEQ ID NOs: 2-25, a solid substrate to which the at least one peptide is attached, and a detectably labeled antibody that specifically binds to IgE or IgD, wherein the IgE or IgD are optionally human; optionally, wherein the at least one peptide comprises or consists of any one of SEQ ID NOs: 2-20.

39. A kit comprising at least one primer pair capable of specifically amplifying a nucleic acid sequence that encodes a peptide selected from any one of SEQ ID NOs: 2-25, wherein:
(a) at least one primer of the primer pair is detectably labeled; or
(b) the kit further comprises a detectably labeled probe that hybridizes to the nucleic acid sequence amplified by the primer pair;
optionally, wherein the peptide comprises or consists of any one of SEQ ID NOs: 2-20.


SEQUENCE LISTING (38 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 6 aa, Severe acute respiratory syndrome coronavirus 2
NGTHWF

SEQ ID NO: 3 - protein, 9 aa, Severe acute respiratory syndrome coronavirus 2
CDVVIGIVN

SEQ ID NO: 4 - protein, 7 aa, Severe acute respiratory syndrome coronavirus 2
TSGWTFG

SEQ ID NO: 5 - protein, 10 aa, Severe acute respiratory syndrome coronavirus 2
RDLICAQKFN

SEQ ID NO: 6 - protein, 7 aa, Severe acute respiratory syndrome coronavirus 2
NQKLIAN

SEQ ID NO: 7 - protein, 8 aa, Severe acute respiratory syndrome coronavirus 2
TASALGKL

SEQ ID NO: 8 - protein, 6 aa, Severe acute respiratory syndrome coronavirus 2
CLKGCC

SEQ ID NO: 9 - protein, 8 aa, Severe acute respiratory syndrome coronavirus 2
NGIGVTQN

SEQ ID NO: 10 - protein, 13 aa, Severe acute respiratory syndrome coronavirus 2
CTMYICGDSTECS

SEQ ID NO: 11 - protein, 23 aa, Severe acute respiratory syndrome coronavirus 2
CTMYICGDSTECSNLLLQYGSFC

SEQ ID NO: 12 - protein, 10 aa, Severe acute respiratory syndrome coronavirus 2
NLLLQYGSFC

SEQ ID NO: 13 - protein, 16 aa, Severe acute respiratory syndrome coronavirus 2
VYYPDKVFRSSVLHST

SEQ ID NO: 14 - protein, 18 aa, Severe acute respiratory syndrome coronavirus 2
KVCEFQFCNDPFLGVYYH

SEQ ID NO: 15 - protein, 15 aa, Severe acute respiratory syndrome coronavirus 2
LKSFTVEKGIYQTSN

SEQ ID NO: 16 - protein, 12 aa, Severe acute respiratory syndrome coronavirus 2
YNENGTITDAVD

SEQ ID NO: 17 - protein, 19 aa, Severe acute respiratory syndrome coronavirus 2
CPFGEVFNATRFASVYAWN

SEQ ID NO: 18 - protein, 20 aa, Severe acute respiratory syndrome coronavirus 2
LCFTNVYADSFVIRGDEVRQ

SEQ ID NO: 19 - protein, 17 aa, Severe acute respiratory syndrome coronavirus 2
KLPDDFTGCVIAWNSNN

SEQ ID NO: 20 - protein, 22 aa, Severe acute respiratory syndrome coronavirus 2
PYRVVVLSFELLHAPATVCGPK

SEQ ID NO: 21 - protein, 13 aa, Severe acute respiratory syndrome coronavirus 2
CVLGQSKRVDFCG

SEQ ID NO: 22 - protein, 10 aa, Severe acute respiratory syndrome coronavirus 2
NFTTAPAICH

SEQ ID NO: 23 - protein, 14 aa, Severe acute respiratory syndrome coronavirus 2
KPSKRSFIEDLLFN

SEQ ID NO: 24 - protein, 8 aa, Severe acute respiratory syndrome coronavirus 2
KYEQYIKW

SEQ ID NO: 25 - protein, 8 aa, Severe acute respiratory syndrome coronavirus 2
FIEDLLFN

SEQ ID NO: 26 - protein, 22 aa, Severe acute respiratory syndrome coronavirus 2
CASYQTQTNSPRRARSVASQSI

SEQ ID NO: 27 - protein, 18 aa, Unknown
  Description of Unknown SARSr-CoV RaTG13 sequence
CASYQTQTNSRSVASQSI

SEQ ID NO: 28 - protein, 18 aa, Unknown
  Description of Unknown SARS-CoV Urbani sequence
CASYHTVSLLRSTSQKSI

SEQ ID NO: 29 - protein, 18 aa, Unknown
  Description of Unknown SARS-CoV CUHK-W1 sequence
CASYHTVSLLRSTSQKSI

SEQ ID NO: 30 - protein, 18 aa, Unknown
  Description of Unknown SARS-CoV GZ02 sequence
CASYHTVSLLRSTSQKSI

SEQ ID NO: 31 - protein, 18 aa, Unknown
  Description of Unknown SARS-CoV A031 sequence
CASYHTVSSLRSTSQKSI

SEQ ID NO: 32 - protein, 18 aa, Unknown
  Description of Unknown SARS-CoV A022 sequence
CASYHTVSSLRSTSQKSI

SEQ ID NO: 33 - protein, 18 aa, Unknown
  Description of Unknown WIV16 sequence
CASYHTVSSLRSTSQKSI

SEQ ID NO: 34 - protein, 18 aa, Unknown
  Description of Unknown WIV1 sequence
CASYHTVSSLRSTSQKSI

SEQ ID NO: 35 - protein, 18 aa, Unknown
  Description of Unknown SARSr-CoV ZXC21 sequence
CASYHTASILRSTGQKAI

SEQ ID NO: 36 - protein, 18 aa, Unknown
  Description of Unknown SARSr-CoV ZC45 sequence
CASYHTASILRSTSQKAI

SEQ ID NO: 37 - protein, 18 aa, Unknown
  Description of Unknown SARSr-CoV Rp3 sequence
CASYHTASTLRSVGQKSI

SEQ ID NO: 38 - protein, 18 aa, Unknown
  Description of Unknown SARSr-CoV Rs672 sequence
CASYHTASTLRSVGQKSI